(19) United States
(12) Patent Application Publication
(10) Pub. No.: US 2024/0293455 A1
(43) Pub. Date: Sep. 5, 2024

(54) ARMED NK CELLS FOR UNIVERSAL CELL THERAPY

(71) Applicants: Université De Montpellier, Montpellier (FR); Institut National De La Sante Et De La Recherche Medicale (Inserm), Paris (FR); Center Hospitalier Universitaire De Montpellier (Chum), Montpellier Cedex 5 (FR); ICM (Institut De Cancerologie De Montpellier), Montpellier Cedex 5 (FR)

(72) Inventors: Martin Villalba, Montpellier (FR); Christian Jorgensen, St. Clement De Riviere (FR); Bruno Robert, Valflaunes (FR); Pierre Martineau, Montpellier (FR); Francisco Javier Harnandez, Castelnau Le Lez (FR); Jessy Presumey, Montpellier (FR)

(21) Appl. No.: 18/018,687

(22) PCT Filed: Aug. 2, 2021
(86) PCT No.: PCT/EP2021/071586

§ 371 Date: Jan. 30, 2023

(30) Foreign Application Priority Data
Jul. 31, 2020 (FR) FR2008211

(51) Int. Cl. A61K 35/17; A61K 39/00; A61P 35/00; C07K 16/28; C07K 16/32; C12N 5/0783

(52) U.S. Cl. CPC A61K 35/17; A61K 39/4613; A61K 39/464406; A61P 35/00; C07K 16/283; C07K 16/2887; C07K 16/32; C12N 5/0646; C07K 2317/524; C07K 2317/72; C07K 2317/73

(57) ABSTRACT

The present invention relates to the field of therapeutic treatment, particularly of cell therapy based on CD16+ cells and NK (Natural Killer) cells. In particular, the invention relates to a pharmaceutical composition comprising a CD16+ cell, a NK cell or a NK cell precursor, in combination with a recombinant polypeptide comprising a modified Fc region, in particular a modified CH2 domain. More particularly, the invention relates to a composition comprising a CD16+ cell and/or a NK cell, in combination with a recombinant polypeptide capable of binding to the FcγRIII (CD16) surface protein, wherein the recombinant polypeptide is non-covalently bound to the FcγRIII (CD16) surface protein expressed by the CD16+ cell, and wherein said recombinant polypeptide comprises: (i) a modified CH2 domain of a wild-type human IgG1, bound, optionally through a linker, to (ii) a ligand binding domain, wherein the ligand binding domain comprises a sequence capable of binding to a target ligand; wherein the modified CH2 domain is characterized by comprising mutations S239D and I332E with respect to the CH2 domain of a wild-type human IgG1, and wherein said CH2 domain of a wild-type human IgG1 is represented by SEQ ID NO 1, and comprises sequence positions 231-340, according to the EU numbering.

### CROSS-REFERENCE TO RELATED APPLICATIONS

This application is a national stage application filed under 35 U.S.C. § 371 of PCT/EP2021/071586, filed Aug. 2, 2021, which claims the benefit of French Patent Application No. FR2008211, filed Jul. 31, 2020, all of which is incorporated herein, in their entireties, by reference.

### SEQUENCE LISTING

The Sequence Listing associated with this application is filed in electronic format via EFS-Web and is hereby incorporated by reference into the specification in its entirety. The name of the text file containing the Sequence Listing is 12598170401SEQUENCELISTING.TXT. The size of the text file is 21 KB, and the text file was created on Dec. 20, 2021.

### TECHNICAL FIELD

The present invention relates to the field of therapeutic treatment, particularly of cell therapy based on CD16+ cells and/or NK (Natural Killer) cells and/or NK cell precursors. More particularly, the invention relates to a pharmaceutical composition comprising a CD16+ cell, a NK cell or a NK cell precursor, in combination with a recombinant polypeptide comprising a modified Fc region, in particular a modified C_{H}2 domain.

### BACKGROUND ART

The development of cell therapies is in the process of being fully extended to the treatment of progressive diseases such as cancer, serious infections or graft rejection after organ or stem cell transplantation. Most scientific and clinical breakthroughs have been obtained primarily against cancer. Cell therapy has emerged as a very promising therapeutic option against cancer from the clinical successes obtained with T cells genetically modified by means of a chimeric receptor referred to as CAR (chimeric antigen receptor) (CAR-T cells), particularly in the treatment of B cells lymphoproliferative disorders.

At present, CAR-T cells approved by the FDA (Food and Drug Agency) are based on autologous T cells (that come from the patient) genetically modified in certified reference centers before release and re-infusion into the patient. Nevertheless, in a significant number of patients, treatment by means of CAR-T cells is associated with subsequent side effects, even lethal effects (Santomasso B, Bachier C, Westin J, Rezvani K, Shpall E J. The Other Side of CAR T-Cell Therapy: Cytokine Release Syndrome, Neurologic Toxicity, and Financial Burden. Am Soc Clin Oncol Educ Book. 2019; 39: 433-444. doi: 10.1200/EDBK_238691). Moreover, the logistics and cost of this type of treatment are contrary to their wide-scale use. Therefore, so that a larger number of patients can benefit from these types of cell therapies, it is essential to devise alternatives that overcome the drawbacks of CAR-T cells and enable new treatment options to be available.

One of the studied alternatives is the use of NK (Natural Killer) cells: cytotoxic lymphocytes that can destroy target cells without prior antigenic stimulation. Nevertheless, a number of fundamental pre-clinical and clinical outcomes indicate that NK cells alone are not effective for the treatment of different hematological and non-hematological malignant pathologies. Specifically, these NK cells are not effective enough for recognizing their target and/or not cytotoxic enough to generate the desired therapeutic effect. With the aim to increasing their efficacy, these NK cells can be “armed” for recognizing target cells-specific antigens (Sanchez-Martinez et al., Theranostics 8(14):3856-3869, June 2018).

However, there is still a need to have new compositions in cell therapy that do not induce or induce few side effects, are less expensive, highly stable over time and suited for the majority of patients and for the majority of pathologies.

There is also a need to provide new means for the administration of therapeutic compounds, such as therapeutic antibodies and other therapeutic compounds, which can be reliably addressed to the relevant target and with reduced side effects.

An object of the present invention is to fulfill all or part of these needs.

### SUMMARY OF INVENTION

The present invention relates to CD16+ cells, NK cells or NK cell precursors, in combination with a recombinant polypeptide; wherein said recombinant polypeptide comprises (i) a modified Fc region, preferably with at least one modification in a C_{H}2 domain, and (ii) a ligand binding domain, said modified Fc region being capable of binding to said cells or precursors thereof.

As specified in the examples hereinafter, the inventors have noted that CD16+ cells, in particular NK cells, in combination with a recombinant polypeptide capable of binding to a ligand or ligands, remain stable in vitro and in vivo for several days. Furthermore, it has been noted that these armed CD16+ cells, in particular NK cells, were thus capable of specifically targeting target cells through recognition of the appropriate ligand(s).

The CD16 cell surface marker is also referred herein as the FCγRIII surface receptor.

According to one of its objects, the present invention relates to a pharmaceutical composition comprising a CD16+ cell, in combination with a recombinant polypeptide; wherein said recombinant polypeptide comprises (i) a modified Fc (fragment crystallizable) region, and (ii) a ligand binding domain, said Fc region being capable of binding to said CD16+ cell.

According to one of its objects, the present invention also relates to a pharmaceutical composition comprising a NK (Natural Killer) cell or a NK cell precursor, in combination with a recombinant polypeptide; wherein said recombinant polypeptide comprises (i) a modified Fc (fragment crystallizable) region, and (ii) a ligand binding domain, said Fc region being capable of binding to said NK cell or to the NK cell precursor.

The inventors have unexpectedly noted that a NK cell, which is a CD16+ cell, taken from an individual and armed in vitro with a recombinant polypeptide as defined in the present invention, allows increasing NK cell cytotoxicity against target cell. Surprisingly, and contrary to the teachings of Moore et al. 2010, the binding between the Fc receptor of an NK cell and a recombinant polypeptide according to the invention remains stable and specific for several days, particularly for at least 7 days, in vitro and in vivo. Therefore, this combination of an NK cell and a recombinant polypeptide as defined in the present description, which will be referred to as “armed NK cell”, proves to be an effective therapeutic solution against any disease the treatment of which implies the destruction of target cells of interest. This therapeutic solution does not need a genetic modification of the NK cell, unlike NK-CAR cells specifically described in the review of Wang et al. (Luyao Wang, Mei Dou, Qingxia Ma, Ruixue Yao, Jia Liu; Chimeric antigen receptor (CAR)-modified NK cells against cancer: Opportunities and challenges; International Immunopharmacology 74 (2019) 105695), which makes this combination much simpler to use than NK-CAR cells. In fact, a single sample of NK cells, for example taken from cord blood, is sufficient to treat any individual for any and all targeting specificities for which an appropriate ligand can be identified.

The inventors thus provide experimental evidence that CD16+ cells, which may in particular correspond to the Natural Killer cell lineage, can be combined efficiently and in a stable manner with a recombinant polypeptide comprising a modified Fc region.

Further, it has been demonstrated on the present invention that a recombinant polypeptide as disclosed herein has a high affinity for the CD16 receptor, in particular the CD16a receptor, for a long period of time.

The inventors also provide experimental evidence that the attachment to the recited cells is also stable, and even suitable in therapy, when the recombinant polypeptide is bound non-covalently. Accordingly, “binding” or “attachment” of the recited recombinant polypeptide to a CD16+ cell and/or NK cell or NK precursor is preferably considered as “non-covalent”.

The inventors also propose that the corresponding strategy is suitable for administering therapeutic compounds with decreased doses and/or reduced side-effects.

The inventors also propose that the corresponding strategy is suitable for binding, in particular non-covalent binding, of such CD16+ cells and/or NK cells and/or precursors thereof with a plurality of recombinant polypeptides with a modified Fc region, in the sense of the invention.

According to one of these objects, the invention relates to a composition comprising a CD16+ cell in combination with a recombinant polypeptide capable of binding to the FcγRIII (CD16) surface protein, preferably to the FCγRIIIa/CD16a surface protein, wherein the recombinant polypeptide is non-covalently bound to the FcγRIII (CD16) surface protein, preferably to the FCγRIIIa/CD16a surface protein, expressed by the CD16+ cell, and wherein said recombinant polypeptide comprises:
(i) a modified C_{H}2 domain of a wild-type human IgG1, bound, optionally through a linker, to
(ii) a ligand binding domain,
wherein the ligand binding domain comprises a sequence capable of binding to a target ligand; wherein the modified C_{H}2 domain is characterized by comprising mutations S239D and I332E with respect to the C_{H}2 domain of a wild-type human IgG1, and wherein said C_{H}2 domain of a wild-type human IgG1 is represented by SEQ ID NO 1, or by a sequence with a percentage of identity of at least 85% with amino acid sequence SEQ ID NO. 1, and comprises sequence positions 231-340, according to the EU numbering.

According to some embodiments, the C_{H}2 domain of a wild-type human IgG1 is represented by SEQ ID NO. 1. Hence, according to some embodiments, the modified C_{H}2 domain is modified with respect to the C_{H}2 domain of the wild-type human IgG1 represented by SEQ ID NO 1 According to some embodiments, the FcγRIII (CD16) surface protein is FcγRIIIa/CD16a surface protein.

According to some embodiments, the modified C_{H}2 domain comprises at least one additional mutation selected from the list consisting of H268F, S324T, G236A and A330L with respect to the C_{H}2 domain of a wild-type human IgG1.

According to some embodiments, the modified C_{H}2 domain comprises at least one additional mutation selected from the list consisting of H268F, S324T and A330L with respect to the C_{H}2 domain of a wild-type human IgG1.

According to some embodiments, the modified C_{H}2 domain comprises at least one additional mutation selected from the list consisting of H268F and S324T with respect to the C_{H}2 domain of a wild-type human IgG1.

According to some embodiments, the modified C_{H}2 domain comprises amino acid substitutions S239D, I332E and S324T, according to EU numbering, with respect to the C_{H}2 domain of a wild-type human IgG1.

According to some embodiments, the modified C_{H}2 domain comprises amino acid substitutions S239D, I332E and H268F, according to EU numbering, with respect to the C_{H}2 domain of a wild-type human IgG1.

According to some embodiments, the modified C_{H}2 domain comprises amino acid substitutions S239D, I332E and A330L, according to EU numbering, with respect to the C_{H}2 domain of a wild-type human IgG1.

According to some embodiments, the modified C_{H}2 domain comprises amino acid substitutions S239D, I332E, H268F and S324T, according to EU numbering, with respect to the C_{H}2 domain of a wild-type human IgG1.

According to some embodiments, the modification of the C_{H}2 domain with respect to the C_{H}2 domain of a wild-type human IgG1 consists of mutations S239D and I332E.

According to some embodiments, the CD16+ cell is allogeneic with respect to an individual in need thereof.

According to some embodiments, the recombinant polypeptide is an antibody and the modification in the C_{H}2 domain is symmetrical, or asymmetrical, with respect to the pair of C_{H}2 domains (or the pair of heavy chains) constituting the antibody.

According to some embodiments, the recombinant polypeptide comprises a human IgG1 Fc (fragment crystallizable) region comprising the modified C_{H}2 domain.

According to some embodiments, the recombinant polypeptide is an antibody or a fragment thereof comprising the modified CH2 domain as defined herein or the Fc region as defined herein and a ligand binding domain selected from the ligand binding domain of any of the following antibodies: Abagovomab, Abatacept, Abciximab, Abituzumab, Abrilumab, Actoxumab, Adalimumab, Adecatumab, Aducanumab, Aflibercept, Afutuzymab, Alacizumab, Alefacept, Alemtuzumab, Alirocumab, Altumomab, Amatixumab, Anatumomab, Anetumab, Anifromumab, Anrukinzumab, Apolizumab, Arcitumomab, Ascrinvacumab, Aselizumab, Atezolizumab, Atinumab, Altizumab, Atorolimumab, Bapineuzumab, Basiliximab, Bavituximab, Bectumomab, Begelomab, Belatacept, Belimumab, Benralizumab, Bertilimumab, Besilesomab, Bevacizumab, Bezlotoxumab, Biciromab, Bimagrumab, Bimekizumab, Bivatuzumab, Blinatumomab, Blosozumab, Bococizumab, Brentuximab, Briakimumab, Brodalumab, Brolucizumab, Bronticizumab, Canakinumab, Cantuzumab, Caplacizumab, Capromab, Carlumab, Catumaxomab, Cedelizumab, Certolizumab, Cetixumab, Citatuzumab, Cixutumumab, Clazakizumab, Clenoliximab, Clivatuzumab, Codrituzumab, Coltuximab, Conatumumab, Concizumab, Crenezumab, Dacetuzumab, Daclizumab, Dalotuzumab, Dapirolizumab, Daratumumab, Dectrekumab, Demcizumab, Denintuzumab, Denosumab, Derlotixumab, Detumomab, Dinutuximab, Diridavumab, Dorlinomab, Drozitumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab, Enlimomab, Enoblituzumab, Enokizumab, Enoticumab, Ensituximab, Epitumomab, Epratuzomab, Erlizumab, Ertumaxomab, Etanercept, Etaracizumab, Etrolizumab, Evinacumab, Evolocumab, Exbivirumab, Fanolesomab, Faralimomab, Farletuzomab, Fasimumab, Felvizumab, Fezkimumab, Ficlatuzumab, Figitumumab, Firivumab, Flanvotumab, Fletikumab, Fontolizumab, Foralumab, Foravirumab, Fresolimumab, Fulramumab, Futuximab, Galiximab, Ganitumab, Gantenerumab, Gavilimomab, Gemtuzumab, Gevokizumab, Girentuximab, Glembatumumab, Golimumab, Gomiliximab, Guselkumab, Ibalizumab, Ibritumomab, Icrucumab, Idarucizumab, Igovomab, Imalumab, Imciromab, Imgatuzumab, Inclacumab, Indatuximab, Indusatumab, Infliximab, Intetumumab, Inolimomab, Inotuzumab, Ipilimumab, Iratumumab, Isatuximab, Itolizumab, Ixekizumab, Keliximab, Labetuzumab, Lambrolizumab, Lampalizumab, Lebrikizumab, Lemalesomab, Lenzilumab, Lerdelimumab, Lexatumumab, Libivirumab, Lifastuzumab, Ligelizumab, Lilotomab, Lintuzumab, Lirilumab, Lodelcizumab, Lokivetmab, Lorvotuzumab, Lucatumumab, Lulizumab, Lumiliximab, Lumretuzumab, Mapatumumab, Margetuximab, Maslimomab, Mavrilimumab, Matuzumab, Mepolizumab, Metelimumab, Milatuzumab, Minetumomab, Mirvetuximab, Mitumomab, Mogamulizumab, Morolimumab, Motavizumab, Moxetumomab, Muromonab-CD3, Nacolomab, Namilumab, Naptumomab, Narnatumab, Natalizumab, Nebacumab, Necitumumab, Nemolizumab, Nerelimomab, Nesvacumab, Nimotuzumab, Nivolumab, Nofetumomab, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab, Oregovomab, Orticumab, Otelixizumab, Oltertuzumab, Oxelumab, Ozanezumab, Ozoralizumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab, Pintumomab, Polatuzumab, Ponezumab, Priliximab, Pritumumab, Quilizumab, Racotumomab, Radretumab, Rafivirumab, Ralpancizumab, Ramucirumab, Ranibizumab, Raxibacumab, Refanezumab, Regavirumab, Reslizumab, Rilonacept, Rilotumumab, Rinucumab, Rituximab, Robatumumab, Roledumab, Romosozumab, Rontalizumab, Rovelizumab, Ruplizumab, Sacituzumab, Samalizumab, Sarilumab, Satumomab, Secukimumab, Seribantumab, Setoxaximab, Sevirumab, Sibrotuzumab, Sifalimumab, Siltuximab, Siplizumab, Sirukumab, Sofituzumab, Solanezumab, Solitomab, Sonepcizumab, Sontuzumab, Stamulumab, Sulesomab, Suvizumab, Tabalumab, Tacatuzumab, Tadocizumab, Talizumab, Tanezumab, Taplitumomab, Tarextumab, Tefibazumab, Telimomab aritox, Tenatumomab, Teneliximab, Teplizumab, Tesidolumab, TGN 1412, Ticlimumab, Tildrakizumab, Tigatuzumab, TNX-650, Tocilizumab, Toralizumab, Tosatoxumab, Tositumomab, Tovetumab, Tralokimumab, Trastuzumab, TRBS07, Tregalizumab, Tremelimumab, Trevogrumab, Tucotuzumab, Tuvirumab, Ublituximab, Ulocuplumab, Urelumab, Urtoxazumab, Ustekimumab, Vandortuzumab, Vantictumab, Vanucizumab, Vapaliximab, Varlimumab, Vatelizumab, Vedolizumab, Veltuzumab, Vepalimomab, Vesencumab, Visilizumab, Volocixumab, Vorsetuzumab, Votumumab, Zalutumimab, Zanolimumab, Zatuximab, Ziralimumab, Ziv-Aflibercept, and Zolimomab.

According to some embodiments, compositions as defined herein may be for use as a medicament.

According to some preferred embodiments, compositions as defined herein may be for use in a method for treating or preventing a cancer, an autoimmune disease, or an infectious disease in an individual in need thereof.

According to another of these objects, the invention also relates to a pharmaceutical composition comprising the composition as defined herein, optionally further comprising an excipient or a pharmacologically acceptable vehicle.

According to some embodiments, the CD16+ cell is allogeneic with respect to an individual in need thereof may be for use as a medicament.

According to some preferred embodiments, a pharmaceutical composition as defined herein may be for use in a method for treating or preventing a cancer, an autoimmune disease, or an infectious disease in an individual in need thereof.

According to another of these objects, the invention also relates to a composition comprising a NK (Natural Killer) cell in combination with a recombinant polypeptide capable of binding to the FcγRIII (CD16) surface protein, wherein the recombinant polypeptide is non-covalently bound to the FcγRIII (CD16) surface protein expressed by the NK (Natural Killer) cell, and wherein said recombinant polypeptide comprises:
(i) a modified C_{H}2 domain of a wild-type human IgG1, bound, optionally through a linker, to
(ii) a ligand binding domain,
wherein the ligand binding domain comprises a sequence capable of binding to a target ligand;
wherein the modified C_{H}2 domain is characterized by comprising mutations S239D and I332E with respect to the C_{H}2 domain of a wild-type human IgG1, and wherein said C_{H}2 domain of a wild-type human IgG1 is represented by SEQ ID NO. 1, or by a sequence with a percentage of identity of at least 85% with the amino acid sequence SEQ ID NO. 1, and comprises sequence positions 231-340, according to the EU numbering.

According to some embodiments, the CD16 cell is allogeneic with respect to an individual in need thereof.

According to some embodiments, the recombinant polypeptide is an antibody and the modification in the C_{H}2 domain is symmetrical, or asymmetrical, with respect to the pair of C_{H}2 domains (or the pair of heavy chains) constituting the antibody.

According to some embodiments, the recombinant polypeptide comprises a human IgG1 Fc (fragment crystallizable) region comprising the modified C_{H}2 domain.

According to some embodiments, the recombinant polypeptide is an antibody or a fragment thereof comprising the C_{H}2 domain as defined herein, and a ligand binding domain selected from the ligand binding domain of any of the antibodies as defined herein.

According to another of these objects, the invention also relates to a pharmaceutical composition comprising an NK (Natural Killer) cell, in combination with a recombinant polypeptide; wherein said recombinant polypeptide comprises (i) a modified Fc (fragment crystallizable) region, and (ii) a ligand binding domain, said Fc region being capable of binding to said NK cell and comprising at least one modified C_{H}2 domain of a wild-type human IgG1, wherein the modified C_{H}2 domain is characterized by comprising mutations S239D and I332E with respect to the C_{H}2 domain of a wild-type human IgG1, and wherein said C_{H}2 domain of a wild-type human IgG1 is represented by SEQ ID NO 1, or by a sequence with a percentage of identity of at least 85% with the amino acid sequence SEQ ID NO. 1, and comprises sequence positions 231-340, according to the EU numbering.

According to another of these objects, the invention also relates to a NK cell allogeneic with respect to an individual in need thereof attached to a recombinant polypeptide, wherein said recombinant polypeptide comprises (i) a modified Fc (fragment crystallizable) region, and (ii) a binding domain, said Fc region being capable of binding to said NK cell and comprising at least one modified C_{H}2 domain of a wild-type human IgG1 as defined herein; for use as a medicament.

According to another of these objects, the invention also relates to a kit comprising:
a first part comprising a CD16+ cell, preferably a CD16+ cell allogeneic with respect to an individual in need thereof; and
a second part comprising a recombinant polypeptide as defined herein; for use as a medicament.

According to another of these objects, the invention also relates to a kit comprising:
a first part comprising a NK cell, preferably a NK cell allogeneic with respect to an individual in need thereof; and
a second part comprising a recombinant polypeptide as defined herein; for use as a medicament.

According to another of these objects, the invention also relates to a kit comprising:
a first part including a NK cell, preferably a NK cell allogeneic with respect to an individual in need thereof; and
a second part including a recombinant polypeptide comprising (i) a modified Fc (fragment crystallizable) region, and (ii) a ligand binding domain; said Fc region being capable of binding to said NK cell and comprising at least one modified C_{H}2 domain of a wild-type human IgG1 as defined herein; for use as a medicament.

According to another of these objects, the invention also relates to a pharmaceutical composition comprising a CD16+ cell, in combination with a recombinant polypeptide; wherein said recombinant polypeptide comprises (i) a modified Fc (fragment crystallizable) region on the amino acid sequence of the Fc region, and (ii) a ligand binding domain, said Fc region being capable of binding to said CD16+ cell, and further comprising an excipient or a pharmacologically acceptable vehicle.

According to another of these objects, the invention also relates to a pharmaceutical composition comprising a NK (Natural Killer) cell or a NK cell precursor, in combination with a recombinant polypeptide; wherein said recombinant polypeptide comprises (i) a modified Fc (fragment crystallizable) region on the amino acid sequence of the Fc region, and (ii) a ligand binding domain, said Fc region being capable of binding to said NK cell or precursor thereof, and further comprising an excipient or a pharmacologically acceptable vehicle.

According to another one of these objects, the invention also relates to a NK cell allogeneic or autologous with respect to an individual in need thereof, or a NK cell precursor attached to a recombinant polypeptide, wherein said recombinant polypeptide comprises (i) a modified Fc region, capable of binding to said NK cell or precursor thereof, and (ii) a ligand binding domain; for use as a medicament.

According to another one of these objects, the invention relates to a kit comprising:
a first part including a NK cell, preferably allogeneic or autologous with respect to an individual in need thereof, or a NK cell precursor;
a second part including a recombinant polypeptide comprising (i) a modified Fc region and (ii) a ligand binding domain; said Fc region being capable of binding to said NK cell or to a NK cell precursor; for use as a medicament.

According to another one of these objects, the invention also relates to the use of the pharmaceutical composition according to the invention, for use as a medicament.

According to another one of these objects, the invention also relates to the use of the pharmaceutical composition, the NK cell allogeneic or autologous with respect to an individual in need thereof, or NK cell precursor or the kit according to the invention, in a method for treating or preventing a disease wherein it is beneficial to bring said allogeneic or autologous NK cell or NK cell precursor close to a target cell in order to destroy said target cell.

According to another one of these objects, the invention also relates to the use of the pharmaceutical composition, the NK cell allogeneic or autologous with respect to an individual in need thereof, or NK cell precursor or the kit according to the invention, in a method for treating or preventing a cancer, an autoimmune disease and derivatives thereof or an infectious disease in an individual in need thereof.

According to another one of these objects, the invention relates to an in vitro or ex vivo method for preparing a pharmaceutical composition comprising a NK cell or a NK cell precursor, comprising the following steps: a) providing a NK cell, preferably allogeneic or autologous with respect to an individual in need thereof, or a NK cell precursor; b) contacting said NK cell or said NK cell precursor with a recombinant polypeptide, wherein said recombinant polypeptide comprises (i) a modified Fc region, and (ii) a ligand binding domain, said Fc region being capable of binding to said NK cell or to a NK cell precursor.

According to one embodiment, the pharmaceutical composition comprising a NK cell or a NK cell precursor, in combination with a recombinant polypeptide; wherein said recombinant polypeptide comprises (i) a modified Fc (fragment crystallizable) region, and (ii) a ligand binding domain, said Fc region being capable of binding to said NK cell or precursor thereof, can further comprise an excipient or a pharmacologically acceptable vehicle.

According to one embodiment, the modified Fc region can comprise an amino acid sequence of any one of SEQ ID NO. 2 to 13.

According to one embodiment, the modified Fc region can comprise an amino acid sequence having a sequence identity of at least 70% with the amino acid sequence SEQ ID NO. 2, particularly a sequence identity of at least 80% with the amino acid sequence SEQ ID NO. 2, particularly a sequence identity of at least 90% with the amino acid sequence SEQ ID NO. 2, and preferably the modified Fc region comprises an amino acid sequence having 100% identity with SEQ ID NO. 2.

According to one embodiment, the modified Fc region of the recombinant polypeptide can comprise at least one modified C_{H}2 domain.

According to a particular embodiment, the modified C_{H}2 domain of the Fc region of the recombinant polypeptide can comprise an amino acid sequence of any one of SEQ ID NO. 2 to 13 or an amino acid sequence having a sequence identity of at least 70% with the amino acid sequence SEQ ID NO. 2, particularly a sequence identity of at least 80% with the amino acid sequence SEQ ID NO. 2, particularly a sequence identity of at least 90% with the amino acid sequence SEQ ID NO. 2, and preferably the modified C_{H}2 domain comprises an amino acid sequence having 100% identity with SEQ ID NO. 2.

According to one embodiment, the CD16+ cell and/or the NK cell can be allogeneic with respect to an individual in need thereof.

### BRIEF DESCRIPTION OF DRAWINGS

FIG. 1 represents the binding specificity of the Fc SDH, Fc LALA and Fc WT polypeptides and antibodies in combination with CD45⁺/CD56⁺/CD3⁻ eNK cells (in vitro NK cells from cord blood),

FIG. 2 represents the binding stability between the Fc SDH, Fc LALA and Fc WT polypeptides with the Fc receptor (CD16A) of CD16⁺/CD56⁺/CD45⁺/CD3⁻ eNK cells,

FIG. 3 represents the saturating conditions of the Fc receptor on CD45⁺/CD56⁺/CD3⁻ eNK cells by Fc SDH and Fc WT polypeptides,

FIG. 4 represents the cytotoxicity and the degranulation level of the eNK cells armed with Fc SDH or Fc WT antibodies on breast cancer cells (BT20 cell line) survival,

FIG. 5 represents the presence of Fc SDH and Fc WT antibodies (trastuzumab) at the membrane surface of armed eNK cells,

FIG. 6 represents the expression of the CD16 receptor at the surface of eNK cells armed with (i) no Fc, (ii) Fc LALA A647 antibody, (iii) Fc WT A647 antibody or (iv) Fc SDH A647 antibody,

FIG. 7A represents the in vivo binding stability of eNK cells-Fc SDH in a peritoneal fluid sample taken from an adult Swiss-nude mouse,

FIG. 7B represents the in vivo binding stability of eNK cells-Fc SDH in a peritoneal fluid sample taken from an adult Swiss-nude mouse,

FIG. 7C represents the in vivo binding stability of eNK cells-Fc SDH in blood, bone marrow and spleen samples of an adult Swiss-nude mouse,

FIG. 7D represents the in vivo binding stability of eNK cells-Fc SDH in blood, bone marrow and spleen samples of an adult Swiss-nude mouse,

FIG. 8 represents two embodiments illustrating a recombinant polypeptide according to the invention,

FIG. 9A represents the in vitro binding affinity of recombinant Fc polypeptides compared to the Fc WT (wild-type) to CD16a and CD16b receptors,

FIG. 9B represents the kinetics of binding on CD16+ cells of different recombinant Fc polypeptides from 1 h to 72 h after arming,

FIG. 10 represents the simultaneous armament with 2 recombinant Fc SDH antibodies containing on CD16⁺ cells,

FIG. 11 represents the efficacy of ex vivo armed CD16+ cells with a recombinant Fc SDH rituximab on CD20-positive cancer cells.

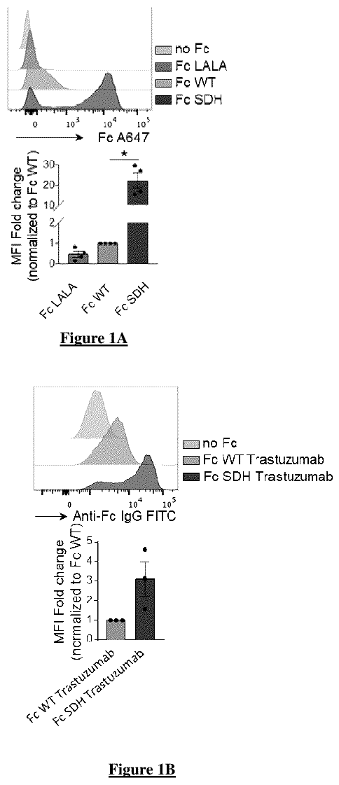

In FIG. 1, which represents the binding specificity of the Fc SDH, Fc LALA and Fc WT polypeptides and antibodies in combination with CD45⁺/CD56⁺/CD3⁻ eNK cells (in vitro NK cells from cord blood): top graph 1A is a histogram representing the results of Fc A647 marker on eNK cells. Y-axis: Number of eNK cells attached to (from top to bottom) (i) no Fc (only cells), (ii) Fc LALA A647, (iii) Fc WT A647, (iv) Fc SDH A647. X-axis: Mean fluorescent intensity (MFI) of A647. Bottom graph 1A. Y-axis: Mean fluorescent intensity (MFI) of modified Fc A647 normalized to Fc WT A647. X-axis: (from left to right) eNK cells armed with the polypeptides (i) Fc LALA A647, (ii) Fc WT A647 and (iii) Fc SDH A647. Representative results from 3 independent experiments using eNK cells from 4 donors. *=p<0.05. One-way ANOVA. Top graph 1B is a histogram representing the results of anti-Fc IgG FITC marker. Y-axis: Number of eNK cells attached to (from top to bottom) (i) no antibody (only cells), (ii) Fc WT trastuzumab antibody and (iiii) Fc SDH trastuzumab antibody. X-axis: Mean fluorescent intensity of FITC. Bottom graph 1B. Y-axis: Mean fluorescent intensity of FITC. X-axis: eNK cells attached to (i) Fc WT trastuzumab antibody and (ii) Fc SDH trastuzumab antibody. Representative results of 2 independent experiments using eNK from 3 donors. Middle graph 1C. Y-axis: Mean fluorescent intensity of A647 marker on eNK cells. X-axis: eNK cells attached to polypeptides (from left to right) (i) Fc LALA A647, (ii) Fc WT A647 or (iii) Fc SDH A647. Right graph 1C. Y-axis: Percentage of Fc⁺ eNK cells in a CD16-eNK cell sample and in a CD16⁺ cell sample. X-axis: eNK cells attached to polypeptides (from left to right) (i) Fc LALA A647, (ii) Fc WT A647 or (iii) Fc SDH A647. Representative results of 3 independent experiments using eNK from of 4 donors. *=p<0.05; ***=p<0.001; ****=p<0.0001; two-way ANOVA.

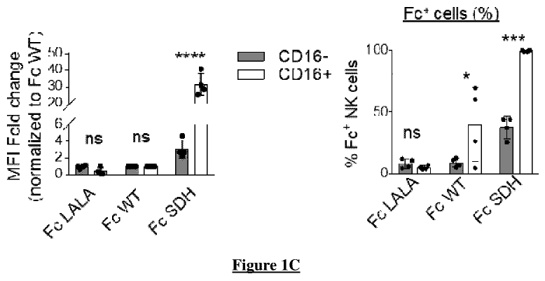

In FIG. 2, which represents the binding stability between the Fc SDH, Fc LALA and Fc WT polypeptides with the Fc receptor (CD16A) of CD16⁺/CD56⁺/CD45⁺/CD3⁻ eNK cells: The results are representative of 3 independent experiments using NK cells from 4 donors on day 2 and 3, 3 donors on day 7. Graph 2A is a histogram representing the results of A647 marker. Y-axis: Number of eNK cells attached to the polypeptides (from top to bottom) (i) no Fc (only cells), (ii) Fc LALA A647, (iii) Fc WT A647, (iv) Fc SDH A647. X-axis: Mean fluorescent intensity of A647. Graph 2B. Y-axis: Percentage of Fc (CD16⁺) eNK cells after 2 days (light grey), 3 days (medium grey) and 7 days (dark grey) of incubation. X-axis: eNK cells attached to polypeptides (from left to right) (i) Fc LALA A647, (ii) Fc WT A647 and (iii) Fc SDH A647. Graph 2C. Y-axis: Mean fluorescent intensity of A647 after 2 days (light grey), 3 days (medium grey) and 7 days (dark grey) of incubation at 37° C. X-axis: eNK cells attached to polypeptides (from left to right) (i) Fc LALA A647, (ii) Fc WT A647 and (iii) Fc SDH A647.

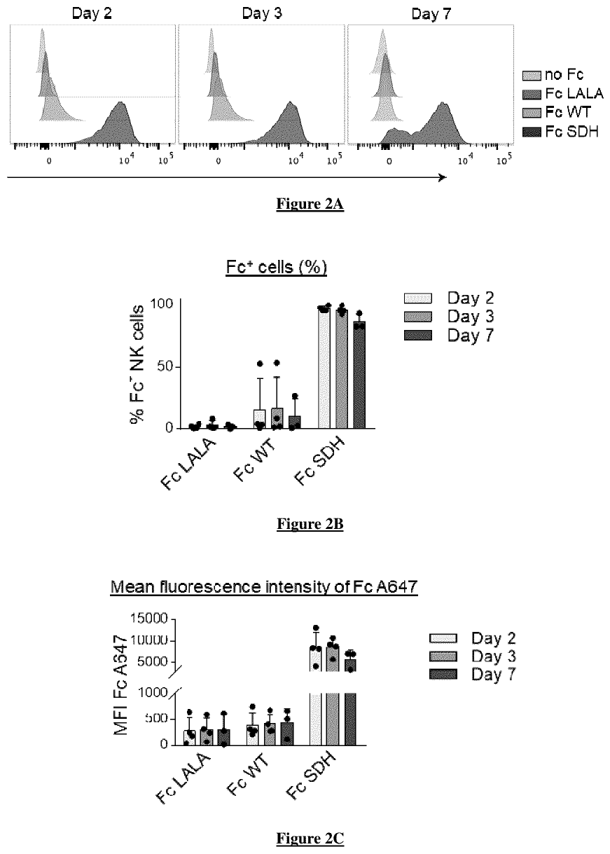

In FIG. 3, which represents the saturating conditions of the Fc receptor on CD45⁺/CD56⁺/CD3-eNK cells by Fc SDH and Fc WT polypeptides: Graph 3A is a histogram representing the saturating conditions of the Fc receptor of eNK cells by Fc WT A647 or Fc SDH A647 polypeptide at 0, 1, 10, 20, 30 or 40 μg/ml. Y-axis: Number of eNK cells attached to (from top to bottom) (i) no Fc (only cells) (ii) 1 μg/ml, (iii) 10 μg/ml, (iv) 20 μg/ml, (v) 30 μg/ml or (vi) 40 μg/ml of Fc WT A647 or Fc SDH A647 polypeptide. X-axis: Mean fluorescent intensity of A647. Graph 3B is a histogram representing the saturating conditions of the Fc receptor of eNK cells by Fc SDH A488 polypeptide at 0, 1, 10, 20, 30 or 40 μg/ml. Y-axis: Number of eNK cells attached to (from top to bottom) (i) no Fc (only cells), (ii) 1 μg/ml, (iii) 10 μg/ml, (iv) 20 μg/ml, (v) 30 μg/ml or (vi) 40 μg/ml of Fc SDH A488. X-axis: Mean fluorescent intensity of A488. The results are representative of one experiment using eNK from one donor (2 pseudoreplicates). Graph 3C is a histogram representing the competing conditions of the Fc receptor, on eNK cells armed with Fc SDH A488 polypeptides, by Fc WT, Fc SDH, Fc block non-labeled competing polypeptides and anti-CD16 antibody at 20 μg/ml. Y-axis: Number of eNK cells armed with Fc SDH A488 polypeptides incubated with (from top to bottom) (i) no Fc (only cells), (ii) no competitor, (iii) Fc block, (iv) Fc WT, (v) Fc SDH and (vi) anti-CD16 antibody at 20 μg/ml for 1 h at 37° C. X-axis: Mean fluorescent intensity (MFI) of A488. Graph 3D is a histogram representing the competing conditions of the Fc receptor of eNK cells armed with Fc SDH A647 polypeptides, by Fc SDH, Fc block non-labeled competing polypeptides and anti-CD16 antibody at 20 μg/ml. Y-axis: Number of eNK cells armed with Fc SDH A647 polypeptides incubated with (from top to bottom) (i) no Fc (only cells), (ii) no competitor, (iii) Fc block, (iv) Fc SDH and (v) anti-CD16 antibody at 20 μg/ml. X-axis: Mean fluorescent intensity (MFI) of A647. The results are representative of one experiment using the eNK from one donor in 2 replicates.

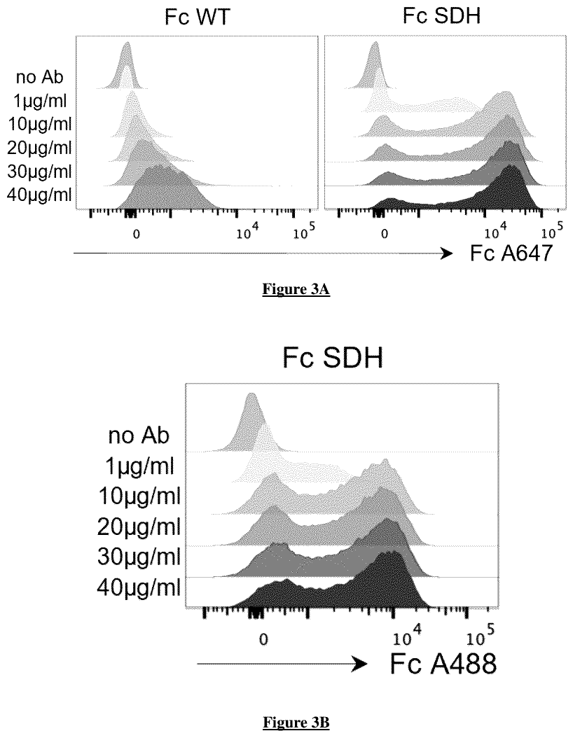

In FIG. 4, which represents the cytotoxicity and the degranulation level of the eNK cells armed with Fc SDH or Fc WT antibodies on the cell survival of cells from breast cancer BT20 cell line: Graph 4A. Y-axis: Cell survival expressed in percentage of BT20 target cells after 1 h of incubation with eNK cells armed for 1 h and which have undergone one washing cycle in order to observe only the armed eNK cells. X-axis: BT20 target cells incubated with (from left to right) (i) no armed eNK cell, (ii) non-armed eNK cells, (iii) eNK cells armed with 10 μg/ml WT antibody, (iv) eNK cells armed with 10 μg/ml SDH antibody, (v) eNK cells armed with 1 μg/ml WT antibody and (vi) eNK cells armed with 1 μg/ml SDH antibody, not washed (that is, there are free antibodies present). Graph 4B. Y-axis: Percentage of cell survival of BT20 cells after 1 h of incubation with eNK cells armed for 1 h and not washed (allows the effect of armed eNK cells but also of free Fc to be observed). X-axis: BT20 target cells incubated with (from left to right) (i) no armed eNK cell, (ii) non-armed eNK cells, (iii) eNK cells armed with 10 μg/ml WT antibody, (iv) eNK cells armed with 10 μg/ml SDH antibody, (v) eNK cells armed with 1 μg/ml WT antibody, (vi) eNK cells armed with 1 μg/ml SDH antibody. The graph is representative of 3 independent experiments using eNK from 4 donors. Graph 4C. Y-axis: Cell survival expressed in percentage of BT20 target cells after 1 h of incubation with eNK cells armed for 24 h. X-axis: BT20 target cells incubated with (from left to right) (i) no armed eNK cell, (ii) non-armed eNK cells, (iii) eNK cells armed with 10 μg/ml WT antibody, (iv) eNK cells armed with 10 μg/ml SDH antibody, (v) eNK cells armed with 1 μg/ml WT antibody and (vi) eNK cells armed with 1 μg/ml SDH antibody. Graph 4D. Armed eNK cells placed in the presence of target cells (bottom graph) or without target cells (top graph 4D). Y-axis: Expression of CD107a at the surface of armed eNK cells, expressed in percentage. X-axis (from left to right): eNK cells armed with (i) no antibody, (ii) 10 μg/ml Fc WT antibody, (iii) 10 μg/ml Fc SDH antibody, (iv) 1 μg/ml Fc WT antibody, (v) 1 μg/ml Fc SDH antibody. The results are representative of two independent experiments using eNK from 3 donors.

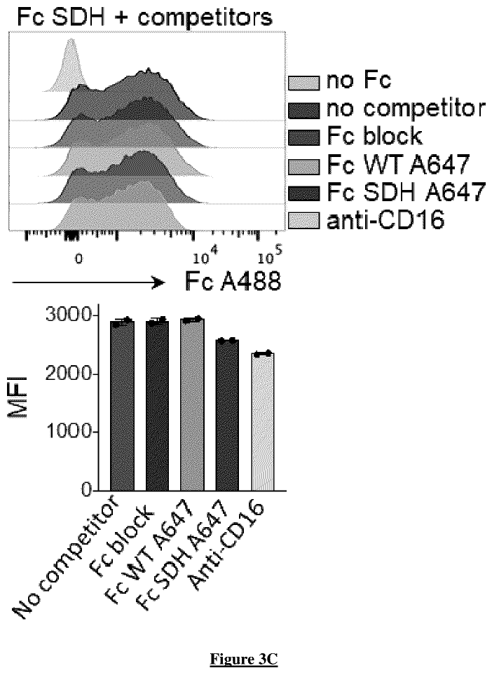

In FIG. 5, which represents the presence of Fc SDH and Fc WT antibodies (trastuzumab) at the membrane surface of armed eNK cells: Graph 5A. Y-axis: Numbers of eNK cells (from top to bottom) (i) in combination with antibodies present at the membrane surface and (ii) in combination with antibodies present in the intracellular domain. X-axis: Mean fluorescent intensity (MFI) of anti-Fc IgG FITC marker attached to (from left to right) (i) no antibody, (ii) Fc WT antibody, (iii) Fc SDH antibody. Graphs 5B and 5C are histograms representing the competing conditions of the Fc receptor (CD16A) of eNK cells armed with the A488 (graph 5B) or A647 (graph 5C) Fc SDH labeled antibodies against (i) the Fc block polypeptide; (ii) anti-CD32 antibody; (iii) anti-CD16 antibody clone B73.1; (iv) anti-CD16 antibody. Y-axis: Number of eNK cells armed with the Fc SDH polypeptide, incubated with (from top to bottom) (i) no Fc, (ii) no competitor, (iii) Fc block polypeptide, (iv) anti-CD32 antibody, (v) anti-CD16 antibody clone B73.1 and (vi) anti-CD16 antibody. X-axis: Mean fluorescent intensity (MFI) of Fc A488 (graph 5B) and Fc A647 (graph 5C). The results are representative of one experiment using eNK cells from one donor (2 pseudoreplicates per donor).

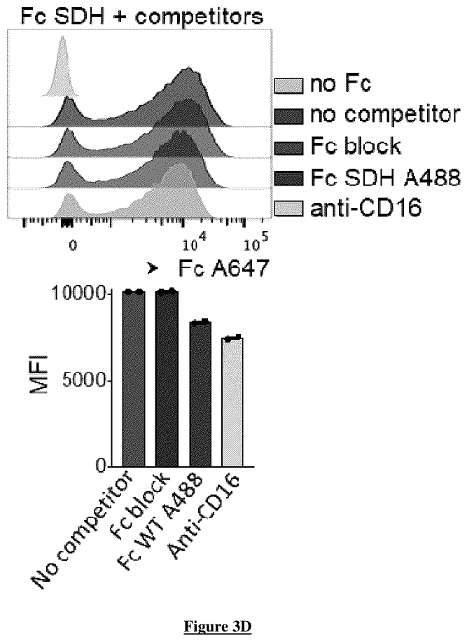

In FIG. 6, which represents the expression of the CD16 receptor at the surface of eNK cells armed with (i) no Fc, (ii) Fc LALA A647 antibody, (iii) Fc WT A647 antibody or (iv) Fc SDH A647 antibody. The results are representative of 3 experiments using eNK cells from one donor. Graph 6A is a histogram representing the results of CD16 surface markers on armed CD56⁺/CD45⁺/CD3⁻ NK cells. Y-axis: Number of eNK cells armed with (from top to bottom) (i) no Fc, (ii) Fc LALA A647 antibody, (iii) Fc WT A647 antibody, (iv) Fc SDH A647 antibody. X-axis: Mean fluorescent intensity of CD16. Graph 6B is a graph representing the expression of CD16 surface markers on armed CD56⁺/CD45⁺/CD3⁻ eNK cells. Y-axis: Expression in percentage of CD16 marker on armed eNK cells. X-axis: eNK cells armed with (from left to right) (i) no antibody Fc, (ii) Fc LALA A647 antibody, (iii) Fc WT A647 antibody, (iv) Fc SDH A647 antibody at 2, 3 and 7 days for each condition.

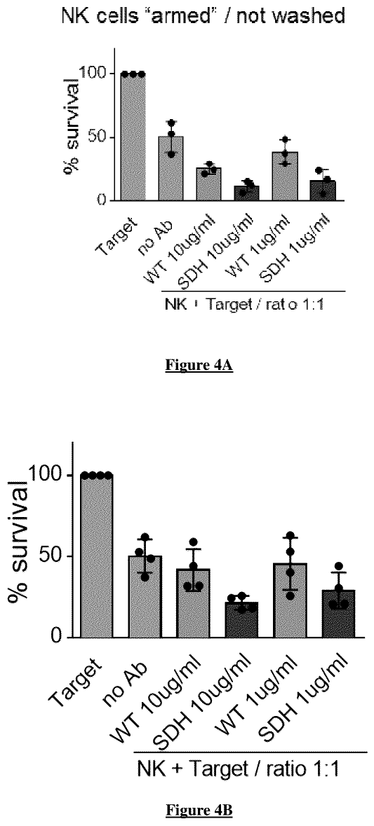

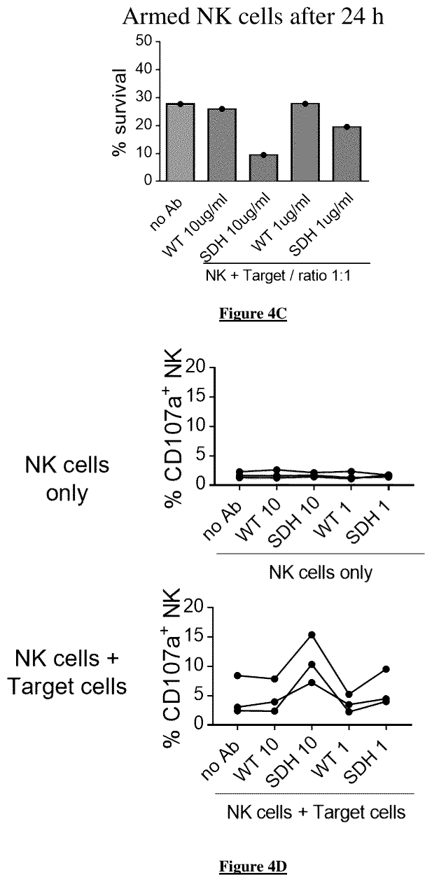

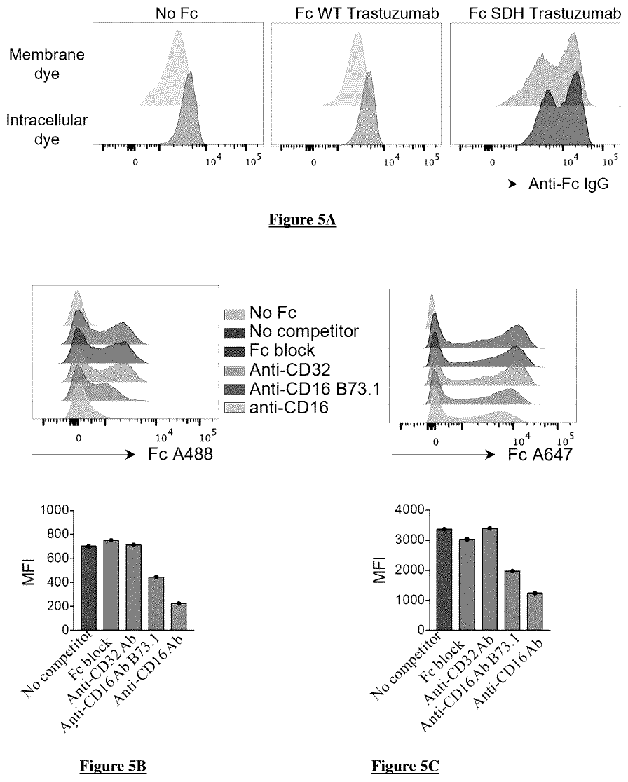

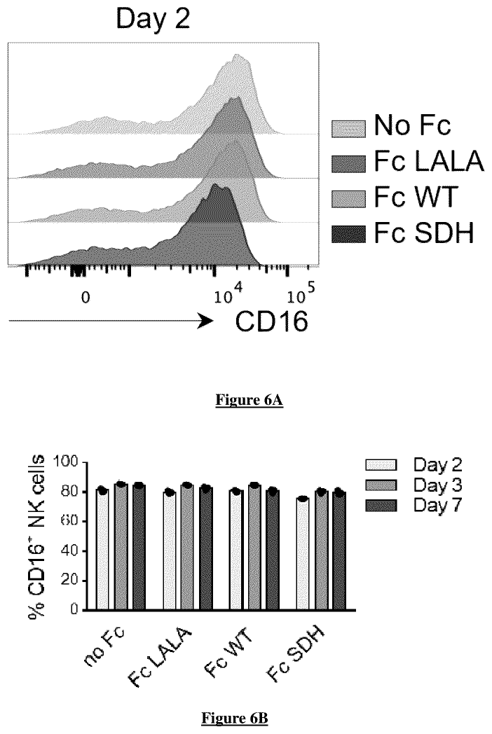

In FIG. 7A, which represents the in vivo binding stability of eNK cells-Fc SDH in a peritoneal fluid sample taken from an adult Swiss-nude mouse: Y-axis: Number of CD45⁺ (left), CD56⁺ (middle), CD16^{-/low} or CD16⁺ (right) armed eNK cells. X-axis: Granulometry measurement of (i) SSC-A (left), CD16 (middle), Fc A647 (right) cells.

In FIG. 7B, which represents the in vivo binding stability of eNK cells-Fc SDH in a peritoneal fluid sample taken from an adult Swiss-nude mouse: Comparison of cells Fc A647⁺ within total CD45⁺/CD56⁺ eNK cells before and after in vivo injection. Y-axis: Number of eNK cells attached to (from top to bottom), (i) no Fc polypeptide, (ii) Fc SDH A647 polypeptide before injection, (iii) Fc SDH A647 polypeptide 24 h after injection. X-axis: Mean fluorescent intensity of A647.

In FIG. 7C, which represents the in vivo binding stability of eNK cells-Fc SDH in blood, bone marrow (graph 7C) and spleen (graph 7D) samples of an adult Swiss-nude mouse: Y-axis: Number of CD45⁺ cells in a blood (left) or bone marrow (right) sample. X-axis: Mean fluorescent intensity of SSC-A.

In FIG. 7D, which represents the in vivo binding stability of eNK cells-Fc SDH in blood, bone marrow (graph 7C) and spleen (graph 7D) samples of an adult Swiss-nude mouse: Y-axis: Number of CD45⁺ (left), and CD16^{-/low} or CD16⁺ A647 labeled (count, right) cells in a spleen sample. X-axis: Mean fluorescent intensity of SSC-A (left), Fc A647 (right).

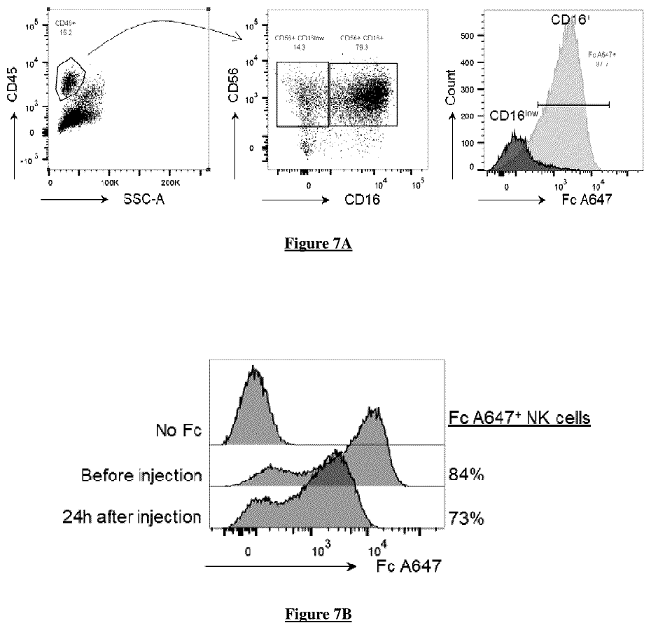

FIG. 8 represents two illustrative embodiments of a recombinant polypeptide according to the invention.

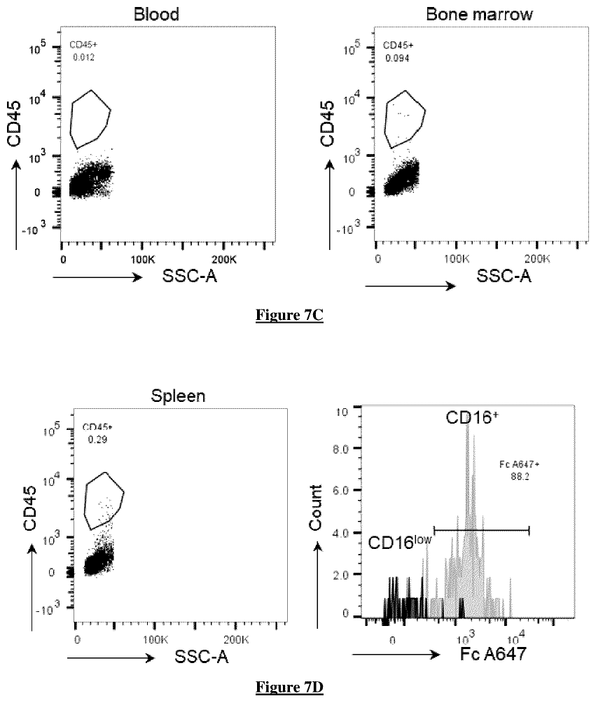

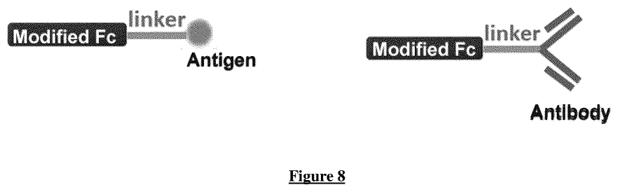

In FIG. 9A, which represents the in vitro binding affinity of recombinant Fc SD (A), Fc IE (B), Fc SDIE (C), Fc AL (D), Fc ALIE (E), Fc GASD (F), Fc GASDALIE (G), Fc GASDIE (H), Fc SDALIE (I), Fc SDHFIE (J), Fc SDSTIE (K) and Fc SDH (L) antibody compared to the Fc WT antibody form to CD16a and CD16b. The graph represents the affinity for CD16a (light grey) and CD16b (dark grey) compared to the reference value the recombinant Fc WT antibody (RTX-WT). Y-axis: Fold improvement of the affinity compared to Fc WT (RTX-WT). X-axis: (from left to right) Fc WT antibody (RTX-WT), Fc SD antibody (RTX A), Fc IE antibody (RTX-B), Fc SDIE antibody (RTX-C), Fc AL antibody (RTX-D), Fc ALIE antibody (RTX-E), Fc GASD antibody (RTX-F), Fc GASDALIE antibody (RTX-G), Fc GASDIE antibody (RTX-H), Fc SDALIE antibody (RTX-I), Fc SDHFIE antibody (RTX-J), Fc SDSTIE antibody (RTX-K) and Fc SDH antibody (RTX-L).

In FIG. 9B which represents the kinetics of binding on CD16+ cells, i.e., eNK cells, of the different recombinant Fc antibodies from 1 h to 72 h after arming the antibodies with CD16+ cells. The graph represents the percent of CD56⁺/CD45⁺/CD16⁺/CD3⁻ eNK cells coated with Fc WT antibody (RTX-WT) or each recombinant Fc antibodies on their surface, at 1 h, 24 h, 48 h or 72 h after arming. Y-axis: Percentage of the frequency of armed eNK cells with each recombinant Fc antibodies. X-axis: eNK cells armed with (from left to right) Fc WT antibody (RTX-WT), Fc SD antibody (RTX A), Fc IE antibody (RTX-B), Fc SDIE antibody (RTX-C), Fc AL antibody (RTX-D), Fc ALIE antibody (RTX-E), Fc GASD antibody (RTX-F), Fc GASDALIE antibody (RTX-G), Fc GASDIE antibody (RTX-H), Fc SDALIE antibody (RTX-I), Fc SDHFIE antibody (RTX-J), Fc SDSTIE antibody (RTX-K), Fc SDH antibody with G1m17,1 allotype (RTX-L-R) and Fc SDH antibody with nG1m1 allotype (RTX-L-T) at 1 h, 24 h, 48 h (except for RTX-L-T, not determined) and 72 h. The graph is representative of 3 independent experiments using NK cells from 4 donors.

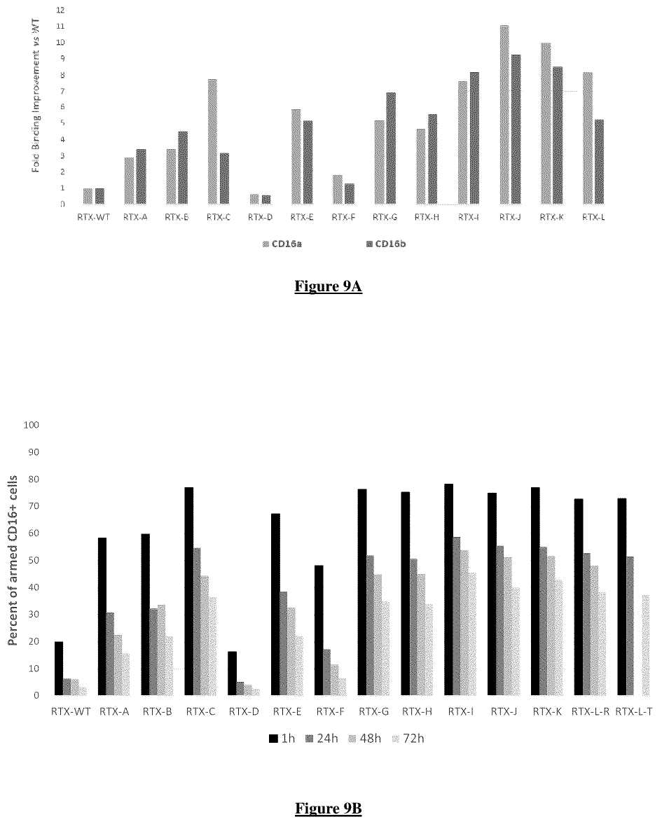

In FIG. 10 which represents the CD16+ cells, i.e., eNK cells, co-armament with two different antibodies containing the Fc SDH mutation in their C_{H}2 domain. CD56⁺/CD45⁺/CD16⁺/CD3⁻ eNK cells were analyzed with anti-trastuzumab antibodies (Y-axis: Number of CD56⁺/CD45⁺/CD16⁺/CD3⁻ eNK cells armed with trastuzumab-Fc SDH) or with anti-rituximab antibodies (X-axis: Number of CD56⁺/CD45⁺/CD16⁺/CD3⁻ eNK cells armed with rituximab-Fc SDH). Left panel: eNK cells were not armed; Middle panels: eNK cells were armed only with RTX-SDH or only with TRAST-SDH; Right panel: eNK cells were armed with both RTX-SDH and TRAST-SDH. RTX-SDH corresponds to recombinant Fc SDH rituximab and TRAST-SDH corresponds to recombinant Fc SDH trastuzumab.

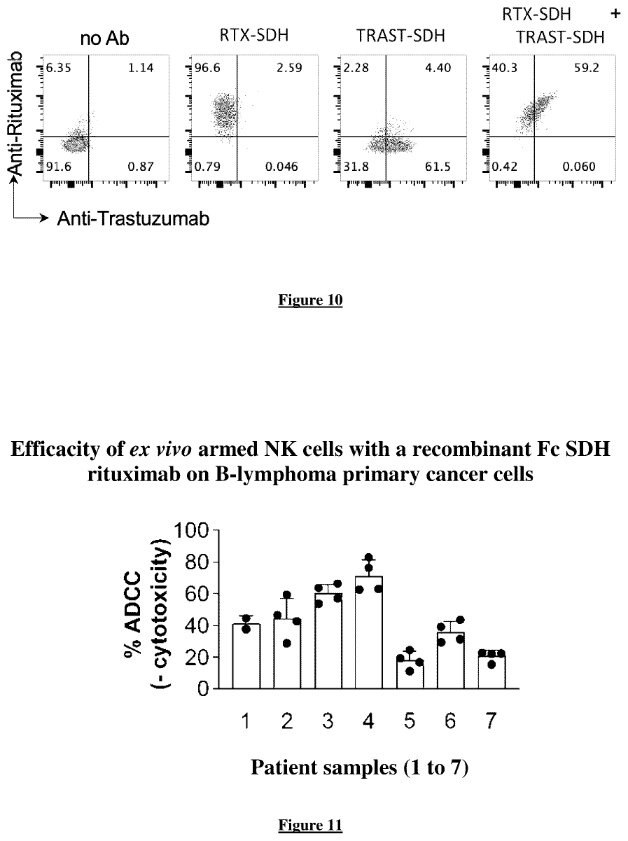

In FIG. 11 which represents the efficacy of ex vivo armed CD16+ cells, i.e., eNK cells, with a recombinant Fc SDH rituximab on CD20+ cancer cells. Y-axis: Percentage of specific antibody-mediated cytotoxicity induced by armed NK cells with RTX-SDH on B-lymphoma primary cancer cells after 16 h of incubation. X-axis: Patient samples from 1 to 7. The graph is representative of 4 independent experiments using NK cells from 4 donors.

### DETAILED DESCRIPTION

As explained in detail in the examples hereinafter, the inventors have noted that an NK cell armed with a recombinant polypeptide comprising (i) a modified Fc (fragment crystallizable) region and (ii) a ligand binding domain, was stable over time under physiological conditions in vitro but also in vivo in mice. Specifically, it was shown that the recombinant polypeptide could not be displaced or replaced under physiological conditions, when the recombinant polypeptide is armed on NK cells. More particularly, the inventors have shown the capacity of these NK cells armed with a recombinant polypeptide according to the invention to induce a strong cytotoxicity against breast cancer BT20 target cells.

In the context of the present invention, the (Natural Killer) NK cell is an example of a CD16+ cell as disclosed herein.

Advantageously, NK cells allogeneic or autologous with respect to an individual in need thereof, according to the invention, can be used for treating or preventing any disease linked to a cell disorder in the body.

The terms used in the present description are used with their common meaning in the technical field under consideration, and with respect to the context of the description in which the terms are used. Certain terms are further discussed hereinafter, or elsewhere in the description, in order to provide additional indications with respect to the invention and the use thereof. The following definitions are provided for the description and the claims.

The description of the different embodiments of the invention comprises the embodiments including “comprising”, “having”, “consisting of” and “consisting essentially of”. The terms “to have” and “to comprise”, or variants such as “has”, “have”, “comprises” or “comprising” should be understood as involving the inclusion of the stated element or elements (such as one element of a composition or one step of a method) but not the exclusion of other elements. The term “consisting of” involves the inclusion of the stated element or elements, excluding any additional element. The expression “consisting essentially of” involves the inclusion of the stated elements, and potentially other elements when the other elements do not substantially affect the novel and fundamental characteristics of the invention. According to the context, the term “comprise” can also strictly involve the stated characteristics, integers, steps or components and, consequently, in this case, it can be replaced with “consist of”.

The term “about” or “approximately” as used herein with respect to a numerical value, refers to the common error interval for the value under consideration, as usually identified by those skilled in the art in the technical field under consideration. The use of the term “about” with respect to a specific value or parameter includes and describes as such this value or this parameter. The term “about” refers to ±10% of a given value. However, every time the value in question refers to a non-divisible object that would lose its identity if further divided, then “about” refers to ±1 of the non-divisible object.

The term “individual” or “patient” as used herein particularly designates a mammal. The considered mammals include, but are not limited to, domestic animals (for example, bovine, ovine, cats, dogs, and horses), primates (for example human and non-human primates), rabbits and rodents (for example, mice and rats). According to a particular embodiment, an individual, or patient, is a human being.

In the context of the present invention, the terms “prevent”, “prevention” (and variants of these expressions) with respect to a physiological disorder or to a disease refers to the prophylactic treatment of the disease or disorder, for example in an individual suspected of having this disease or disorder, or at risk of developing this disease or disorder. Preventing includes, but is not limited to, the prevention or slowing down of the development of the disease, and/or the maintaining of one or several symptoms of the disease to a desired level or a lesser level. The term “prevent” does not require 100% elimination of the likelihood or the probability of occurrence of the disease or disorder.

Rather, this term designates the reduction to a lesser degree of the risk or the probability of occurrence of a given phenomenon. As indicated, the prevention can be complete, that is, the absence of detectable symptoms or disease, or partial, such that there are fewer symptoms, or the symptoms are less severe.

In the context of the present invention, the terms “therapeutically effective amount” and “prophylactically effective amount” refer to an amount that provides a therapeutic advantage in the treatment, prevention or management of the pathologic conditions under consideration. The specific amount that is therapeutically effective can readily be determined by a doctor and can vary depending on factors such as the type and the stage of the pathologic conditions under consideration, medical history, sex, weight and age of the patient, the patient's diet, and the administration of other therapeutic agents.

In the sense of the invention, the term “significantly” or any derived term, used in the context of a change, means that the observed change is notable or has statistical significance.

In the context of the present invention, the terms “treating”, “treatment”, “therapy” or “therapeutic” refer to the administration or consumption of an active ingredient, that is an armed NK cell according to the invention, or of a pharmaceutical composition comprising such an active ingredient for the purposes of curing, relieving, reducing or attenuating, or improving a disease or a pathological disorder, or one or several associated symptoms, or for preventing or slowing down the progression of this symptom or symptoms or this disease, or for stopping the development of this symptom or symptoms, or this disease or this pathological disorder, in a statistically significant manner. More particularly, “treating” or “treatment” includes any approach for obtaining a beneficial effect or a desired result with respect to a disease in an individual. The beneficial or desired clinical results can include, but are not limited to, the attenuation or improvement of the disease or one or several symptoms of such a disease; the diminution or reduction of the extent of the disease, the stabilization, that is, the absence of worsening of a disease, or one or several symptoms of such a disease; the prevention of a disease, or one or several symptoms of such a disease; the prevention of the propagation of a disease, or one or several symptoms of such a disease; the slowing down of a disease, or one or several symptoms of such a disease or the progression of one or more symptoms of such a disease; the diminution of the recurrence of an associated disease, or one or several symptoms of such a disease; and the interruption of a disease, or one or several symptoms of such a disease. In other words, “treatment” as used herein comprises any recovery, improvement, reduction or interruption of a disease, or one or several symptoms of such a disease. A “reduction” of a symptom or a disease means a diminution of the severity or the frequency of the disease or the symptom, or the elimination of the disease or the symptom.

As used in the present description and the claims, the singular forms “a”, “an”, and “the” include the plural forms, unless otherwise and explicitly indicated in the context.

“Pharmaceutically acceptable” or “physiologically acceptable” means that the vehicle (carrier, diluent, or excipient) should be compatible with the other ingredients of the formulation and non-toxic for the individual to whom the composition comprising same is to be administered. A pharmaceutically acceptable vehicle is a vehicle known as satisfactory, specifically, according to the criteria of safety, compatibility, and inertness required for use in the pharmaceutical field. By way of example of pharmaceutically acceptable vehicle, sterile water, saccharides such as sucrose or saccharose, starches, sugar alcohols such as sorbitol, polymers such as PVP or PEG, lubricating agents, such as magnesium stearate, preservatives, dyeing agents or flavors can be mentioned.

In the context of the present invention, the expression “physiologically acceptable vehicle” aims to designate any substance or composition compatible with the organism of the individual to whom an active ingredient of the invention is to be administered. Particularly, a physiologically acceptable vehicle is a substance or composition the administration of which to an individual does not come with significant deleterious effects. It can be, for example, a non-toxic solvent such as water or a saline aqueous solution. Particularly, such a vehicle is compatible with oral or rectal administration, and is preferably adapted to oral administration.

The list of sources, ingredients and components indicated hereinafter are understood to be described such that any combination and mixture thereof is also contemplated in the context of the present invention.

Any maximal numerical limitation given in the description is understood to comprise any lower numerical limitation, as if these lower numerical limitations were expressly written. Any minimal numerical limitation given in this description comprises any higher numerical limitation, as if these higher numerical limitations where expressly written herein. Any numerical interval given throughout the description comprises any narrower numerical interval included in such a wider numerical interval, as if these narrower numerical intervals were all expressly written.

Any list indicated in the description, such as, for example, the lists of ingredients, is intended and should be construed as Markush groups. Therefore, any list can be read and construed as elements “selected from the group consisting of” . . . list of elements . . . “and the combinations and mixtures thereof”.

Reference can be made hereinafter to trade names of components comprising several ingredients used in the present description. The inventors do not intend to be limited to materials with a specific trade name. Materials (for example, those obtained from a different source with a different name or reference number) equivalent to those indicated herein by means of a trade name can be substituted and used in the description hereinafter.

The term “parenteral administration” designates administration routes other than enteral and topical administration, generally by injection, by intravenous, intramuscular, intraarterial, intrathecal, intracapsular, intraorbital, intracardiac, intradermal, intraperitoneal, transtracheal, subcutaneous, epidermal, intraarticular, subcapsular, subarachnoid, intraspinal, epidural and intrasternal routes, including, without limitation, injections and perfusions.

In the context of the present invention, “identity percentage” between two nucleic acid or amino acid sequences in the sense of the present invention, means to designate a percentage of nucleotides or amino acid residues which are identical between the two sequences to be compared, obtained after the best alignment (optimal alignment), this percentage being merely statistical and the differences between the two sequences being randomly distributed throughout their length. Sequence comparisons between two nucleic acid or amino acid sequences are conventionally carried out comparing these sequences after having been optimally aligned; said comparison can be performed by segments or by a “comparison window”. Optimal alignment of the sequences for comparison can be performed, other than manually, by means of comparison software such as BLAST. Thus, a “sequence identity percentage of at least about 70%” specifically includes a sequence identity percentage of at least 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% 98%, 99%.

As used herein, “+”, when used to indicate the presence of a particular cellular marker, means that the cellular marker is detectable by fluorescence activated cell sorting over an isotype control or is detectable above background in quantitative or semi-quantitative RT-PCR or using any other well-known technics allowing marker detection.

In the context of the present invention, “CD16⁺ cells” means a cell or population of cells that is “+”/“positive” for FcγRIII/CD16 and refers to the detectable presence on or in the cell of FcγRIII/CD16. CD16⁺ cell from any origin, preferably mammalian and more preferably human. “CD16⁺ cell” also refers to unmodified cells naturally expressing FcγRIII/CD16 or transiently modified cells to allow expression or overexpression of FcγRIII/CD16 or stably transformed cells to express or overexpress the FcγRIII/CD16. The transient or stable modifications refer to well-known modifications from the scientific knowledge such as but not limited to expose cells to chemical or biological reagent(s) or to genetically modified cells by transfecting nucleotides agents). For instance, the term “CD16+ cells” encompasses natural killer (NK) cells, neutrophils, monocytes, dendritic cells and macrophages.

In the context of the present invention, “armed CD16⁺ cells” means a CD16⁺ cell, preferably allogeneic or autologous with respect to an individual in need thereof, more preferably allogeneic with respect to an individual in need thereof, in combination with a recombinant polypeptide; comprising (i) a modified Fc region or a variant thereof, preferably comprising a modified C_{H}2 domain, and (ii) a ligand binding domain.

“Armed CD16⁺ cells” means a compound of construct (C) such as: [CD16+ cell]-[Recombinant polypeptide of formula (I)]. Therefore, “armed CD16⁺ cell” encompasses a compound of construct (D): [CD16⁺ cell]-[[Modified Fc region]-[Linker]ₓ-[Ligand binding domain]].

In each of the constructs C or D, the recombinant polypeptide of formula (I) is not attached to the CD16⁺ cell by a covalent bond. On the contrary, in each of the constructs (C) or (D) the peptide of formula (I) is attached to the CD16⁺ cell in a non-covalent manner, particularly by an association of the receptor/receptor ligand type.

In the context of the present invention, an “antibody” or “antigen binding polypeptide” refers to a polypeptide or polypeptide complex that specifically recognizes and binds to one or more antigens. An antibody can be a whole antibody or any antigen binding fragment or single chain thereof. Thus, the term “antibody” includes any protein or peptide comprising at least a portion of an immunoglobulin molecule which has biological activity for binding to an antigen. An example includes, but is not limited to, the complementarity determining region (CDR) of the heavy/light chain or its ligand binding portion, the heavy or light chain variable region, the heavy or light chain constant region, the framework (FR) region or any portion thereof, or at least a portion of binding protein. The term antibody also encompasses a polypeptide or polypeptide complex that possesses antigen binding ability upon activation. The term “antibody fragment” or “antigen-binding fragment” as used herein is part of an antibody, such as F(ab′) 2, F(ab) 2, Fab′, Fab, Fv, single-chain variable fragment (scFv), and the like; for example diabodies. Regardless of the structure, the antibody fragment binds to the same antigen recognized by the intact antibody.

Other ligand binding domains, and alternatives to antibody fragment” may include nucleic-acid based ligands such as aptamers and spiegelmers.

The term “antibody fragment” also includes any synthetic or genetically engineered protein, or polypeptide, that acts like an antibody by binding to a specific antigen to form a complex. An antibody, an antigen binding polypeptide, or a variant or derivative thereof according to the invention includes, but is not limited to, a polyclonal antibody, monoclonal antibody, multispecific antibody, human antibody, humanized antibody, primatized antibody or chimeric antibody, single-chain antibody, epitope-binding fragment (e.g., Fab, Fab′ and F(ab′) 2, Fd, Fvs, single-chain Fvs (scFv)), single-chain antibody, disulfide-linked Fvs (sdFv), fragment comprising a VK or VH domain, fragment generated from a Fab expression library, and anti-Id antibody (including, for example, the anti-Id antibody and LIGHT antibody disclosed herein). The immunoglobulin or antibody molecule of the invention may be an immunoglobulin molecule of any type (e.g., IgG, IgE, IgM, IgD, IgA, and IgY), species (e.g., IgG1, IgG2, IgG3, IgG4, IgA1, and IgA2) or subtype.

In the context of the present invention, “ligand binding domain from an antibody” refers to a sequence capable of binding to a target ligand, such as a target substance, a target compound, a target molecule or even a target cell, (i) for example a ligand binding domain which is capable of binding to a specific antigen of a target cell, or a sequence capable of binding to a natural or tumor antigen, such as the antigen binding domain of an antibody, which is capable of binding to said target or (ii) for example a ligand binding domain which is capable of binding to a cell receptor or binding to the antigen binding domain of an antibody, in which case the ligand binding domain comprises, or consists of, an antigen recognized by the antigen binding domain of said antibodies.

The term “antigen binding fragment” or ‘antigen binding domain” or ‘ligand binding domain from an antibody”, as used herein, refers in particular to one or more fragments of an intact antibody that retain the ability to specifically binds to a given antigen/ligand. Antigen binding functions of an antibody can be performed by fragments of an intact antibody. Examples of binding fragments encompassed within the term antigen binding fragment of an antibody include a Fab fragment, a monovalent fragment consisting of the VL, VH, CL and CH1 domains; a Fab′ fragment, a monovalent fragment consisting of the VL, VH, CL, CH1 domains and hinge region; a F(ab′)₂ fragment, a bivalent fragment comprising two Fab′ fragments linked by a disulfide bridge at the hinge region; an Fd fragment consisting of VH domains of a single arm of an antibody; a single domain antibody (sdAb) fragment (Ward et al., 1989 Nature 341:544-546), which consists of a VH domain or a VL domain; and an isolated complementary determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv); see, e.g., Bird et al., 1989 Science 242:423-426; and Huston et al., 1988 proc. Natl. Acad. Sci. 85:5879-5883). “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody. These antibody fragments are obtained using conventional techniques known to those skilled in the art, and the fragments are screened for utility in the same manner as are intact antibodies. A unibody is another type of antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies. Antigen binding fragments can be incorporated into single domain antibodies, SMIP, maxibodies, minibodies, intrabodies, diabodies, triabodies and tetrabodies (see, e.g., Hollinger and Hudson, 2005, Nature Biotechnology, 23, 9, 1126-1136). The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Antigen binding fragments can be incorporated into single chain molecules comprising a pair of tandem Fv segments (VH-CH1-VH-CH1) Which, together with complementary light chain polypeptides, form a pair of antigen binding regions (Zapata et al., 1995 Protein Eng. 8(10); 1057-1062 and U.S. Pat. No. 5,641,870).

The Fab of the present invention can be obtained by treating an antibody which specifically reacts with a target antigen/ligand with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained by treating an antibody which specifically reacts with a target antigen with a protease, pepsin. Also, the F(ab′)2 can be produced by binding Fab′ described below via a thioether bond or a disulfide bond.

The Fab′ of the present invention can be obtained by treating F(ab′)2 which specifically reacts with a target antigen with a reducing agent, dithiothreitol.

In the context of the present invention, the “FcγRIIIa/CD16a surface protein” refers to the activating receptor CD16a, also known as FcγRIIIA, mainly expressed on the cell surface of immune cells. CD16a is an activating receptor triggering the antibody-dependent cytotoxic activity of immune cells. For instance, FcγRIIIa/CD16a surface protein cells encompasse natural killer (NK) cells, subsets of monocytes, dendritic cells, and rare T cells.

In the context of the present invention, the “FcγRIIIb/CD16b surface protein” refers to the activating receptor CD16B, also known as FcγRIIIB, mainly expressed on the cell surface of white blood cells. For instance, FcγRIIIb/CD16b surface protein cells encompass NK cells and neutrophil cells.

In the context of the present invention, “armed NK cell” means a NK cell, preferably allogeneic or autologous with respect to an individual in need thereof, in combination with a recombinant polypeptide; comprising (i) a modified Fc region or a variant thereof, preferably comprising a modified C_{H}2 domain, and (ii) a ligand binding domain.

“Armed NK cell” means a compound of construct (A) such as: [NK cell]-[Recombinant polypeptide of formula (I)]. Therefore, “armed NK cell” encompasses a compound of construct (B): [NK cell]-[[Modified Fc region]-[Linker]ₓ-[Ligand binding domain]].

In each of the constructs A or B, the recombinant polypeptide of formula (I) is not attached to the NK cell by a covalent bond. On the contrary, in each of the constructs (A) or (B) the peptide of formula (I) is attached to the NK cell in a non-covalent manner, particularly by an association of the receptor/receptor ligand type.

In a polypeptide of formula (I), comprising a “ligand binding domain” unit, said “ligand” can be of any type, as long as (i) said ligand binding domain comprised in the recombinant polypeptide of formula (I) is capable of binding said ligand.

In certain embodiments of a polypeptide of formula (I), the ligand binding domain can consist of a molecule recognized by a receptor, for example a molecule recognized by a receptor expressed by a target cell, or even a molecule recognized by the antigen binding domain of an antibody. In the last case, the ligand binding domain comprised in a recombinant polypeptide of formula (I), which is recognized by the antigen binding domain of an antibody, can also be referred to as “antigen”.

In other embodiments of a polypeptide of formula (I), the ligand binding domain comprises an antigen binding domain of an antibody. In these other embodiments, the ligand is a molecule recognized by said antibody binding domain, such as a molecule expressed by a target cell, for example a target tumor antigen, a target cell marker protein, or even a target cell receptor.

The term “antibody” used herein is a specific form of a polypeptide comprising an Fc domain comprising at least one ligand binding to a domain containing, or conserving, substantial homology with at least one of the variable domains of a heavy or light chain antibody of at least one kind of animal antibody. The constant sequences of the wild-type human IgG sub-class are classified in the UniProt database available on-line under the names P01857 (IgG1), P01859 (IgG2), P01860 (IgG3) and P01861 (IgG4). Herein, the expression “Fc region of wild-type human IgG1s” refers to the Fc region of human IgG1s that can be illustrated by the amino acid sequence of SEQ ID NO. 15 or a fragment of this SEQ ID NO. 15.

Herein, the expression “Fc region of wild-type human IgG1s” also may refer to the Fc region of human IgG1s that can be illustrated by the amino acid sequence of SEQ ID NO. 16 or a fragment of this SEQ ID NO. 16.

Herein, the expression “C_{H}2 domain of a wild-type human IgG1” relates to a part of the Fc region of human IgG1s that can be illustrated by the amino acid sequence of SEQ ID NO. 1 or a fragment of this SEQ ID NO. 1.

Specifically, SEQ ID NO. 15 or SEQ ID NO. 16 comprise SEQ ID NO. 1. SEQ ID NO 16. comprises SEQ ID NO. 17.

“Ligand of Fc region of an IgG” means a molecule, preferably a polypeptide, suitable for binding the Fc region of an IgG-type, particularly of an IgG1-type, antibody to form a non-covalent complex. In a non-limiting manner, such Fc ligands include the polypeptides FcγRs, FcRn, C1q, C3, mannan-binding lectin, mannose receptor, staphylococcal protein A, streptococcal protein G, and viral origin FcγR. Fc ligands particularly include the Fc receptor homologs (FcRH), (Davis et al., 2002, Immunological Reviews 190:123-136).

“Fcγ receptor” or “Fc receptor” or “FcγR”, or “FcgammaR”, mean any member of the protein family encoded by an FcγR gene and suitable for binding the Fc region of an IgG-type, and more particularly of an IgG1-type, antibody. In humans, this family includes, without limitation, FcγRI (CD64), which includes FcγRIa, FcγRIb, and FcγRIc isoforms; FcγRII (CD32), including FcγRIIa (including the H131 and R131 allotypes), FcγRIIb (including FcγRIIb-1 and FcγRIIb-2), and FcγRIIc isoforms; and FcγRIII (CD16), including FcγRIIIa (including V158 and F158 allotypes) and FcγRIIIb (including FcγRIIb-NA1 and FcγRIIb-NA2 allotypes) isoforms. An FcγR can be from any kind of organism, including specifically humans, mice, rats and monkeys.

Most particularly the FcγR receptors suitable for recognizing a modified Fc region according to the invention are the FcγRIII (CD16) receptors and isoforms thereof.

“Fc region” means all or part of an antibody Fc fragment, or “fragment crystallizable region (Fc region)”, which generally consists of the heavy chain constant portion beyond the hinge portion, comprising a C_{H}2 and C_{H}3 domain, that is, respectively, “heavy chain constant domain 2” and “heavy chain constant domain 3”. Also, this term encompasses the last two constant regions of IgA, IgD and IgE-type immunoglobulins, the last three constant regions of IgM and IgE-type immunoglobulins as well as the N-terminal hinge portion of said regions.

Particularly, it means all or part of an Fc fragment of a human IgG-type antibody, and most particularly of an IgG1-type antibody.

According to certain particular embodiments, said Fc region can comprise all or part of a C_{H}2-C_{H}3 region, of a C_{H}2 region, or of a C_{H}3 region. “Modified Fc region”, or “variant Fc region”, means a polypeptide sequence corresponding to a modified form of a reference Fc region, such as a reference Fc region of a human IgG1. Therefore, a modified Fc region in the sense of the invention differs from a reference sequence of an antibody Fc fragment, particularly of a human IgG1 Fc fragment, by one or several amino acid modifications. Such a modified region can relate indiscriminately to a recombinant polypeptide, to a composition (for example, pharmaceutical composition) comprising said recombinant polypeptide.

According to certain embodiments, said modified Fc region has at least one amino acid modification with respect to said reference sequence (particularly said reference sequence of human IgG1), which encompasses at least one, two, three, four, five, or more than five modifications with respect to said reference sequence. According to certain embodiments, said modified Fc region comprises at least 70% sequence identity with respect to said reference (particularly, said reference sequence of human IgG1).

According to preferred embodiments, said modified Fc region has a modulated affinity with respect to the FcγRIII (CD16) surface protein expressed by the CD16+ cells.

According to preferred embodiments, said modified Fc region has an increased or decreased affinity with respect to the FcγRIII (CD16) surface protein expressed by the CD16+ cells.

According to certain preferred and exemplary embodiments, said modified Fc region has a modulated affinity with respect to the FcγRIII (CD16) surface protein expressed by the NK cells, or, where applicable, any precursor thereof.

Particularly, according to some of these embodiments, said modified Fc region has an increased affinity with respect to the FcγRIII (CD16) surface protein expressed by the NK cells, or, where applicable, any precursor thereof.

According to certain alternative embodiments, said modified Fc region has a decreased affinity with respect to the FcγRIII (CD16) surface protein expressed by the NK cells, or, where applicable, any precursor thereof.

For all the positions discussed in the context of the previous invention, and relative to modifications within the Fc region, such as those defined in sequences SEQ ID NO. 2 to 14, with respect to SEQ ID NO. 1, and unless otherwise indicated, reference will be made to EU numbering by reference to Edelman (Edelman et al., 1969, Proc Natl Acad Sci USA 63:78-85, as incorporated by reference). Particularly, said modification can be an addition, a deletion or a substitution. A substitution can include specifically any natural or non-natural amino acid.

A polypeptide sequence comprising “1 to 100 amino acids” includes, specifically, an amino acid sequence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100 amino acids.

In the context of the present invention, “modification of an amino acid” means a substitution, an insertion and/or a deletion of an amino acid in a polypeptide sequence. “Amino acid substitution” or “substitution” means the replacement of an amino acid in a particular position in a wild-type polypeptide sequence with another amino acid. For example, the S239D substitution refers to a variant polypeptide, in this case an Fc variant, wherein serine in position 239 is replaced with aspartic acid. “Insertion of an amino acid” or “insertion” in the sense of the present document, the addition of an amino acid to a specific location in a parental polypeptide sequence. For example, the G>235-236 insert designates an insertion of glycine between positions 235 and 236. “Deletion of an amino acid” or “deletion” herein means the elimination of an amino acid at a level in a parental polypeptide sequence. For example, G236 designates the deletion of glycine in position 236.

“Position” herein means a location in a protein or a polypeptide sequence. The positions can be numbered sequentially or according to an established format, for example the EU index such as in Kabat. For any position approached in the present invention, the numbering is according to EU numbering relating to the numbering of the EU antibody (Edelman et al., 1969, Proc Natl Acad Sci USA 63:78-85, herein incorporated by reference in its entirety). Correspondence with the Kabat numbering can be established by means of the correspondence table; IMGT unique numbering for C-DOMAIN (http://www.imgt.org/IMGTScientificChart/Numbering/Hu_IGHGnber.html).

“IgG” herein means a polypeptide belonging to the class of antibodies which are essentially encoded by a recognized gamma immunoglobulin gene. In humans, this IgG comprises the IgG1, IgG2, IgG3 and IgG4 sub-classes or isotypes. In mice, the IgGs comprise IgG1, IgG2a, IgG2b, IgG3. “Isotype” herein means any immunoglobulin sub-class defined by the chemical and antigenic characteristics of its constant regions. The known isotypes of human immunoglobulins are as follows: IgG1, IgG2, IgG3, IgG4, IgA1, IgA2, IgM, IgD and IgE.

“Antibody-dependent cellular cytotoxicity” or “ADCC” designates a form of cytotoxicity wherein the secreted Igs are attached to Fc receptors (FcγR) present on certain cytotoxic (for example, Natural Killer (NK)) cells and allows said cytotoxic cells to bind specifically to an antigen in order to then destroy the target cell with cytotoxins. The IgG antibodies, specific for ligands targeted to the target cell surface, stimulate the cytotoxic cells and are necessary for the killing thereof. Target lysis is carried out extracellularly, requires direct cell-to-cell contact, and does not imply any complement. The capacity of any antibody or polypeptide to mediate target cell lysis by ADCC can be tested. In order to evaluate the ADCC, an antibody of interest is added to the target cells, presenting the target ligand, in combination with effector cells which can be activated by antigen-antibody complexes (for example, NK cells), which leads to target cell cytolysis. Cytolysis is generally detected by the release of markers (for example, radioactive substrates, fluorescent dyes (A647 or A688) or natural intracellular proteins) of lysed cells. Effector cells useful for these analyses comprise peripheral blood mononuclear cells (PBMC) and NK cells. Specific examples of in vitro ADCC tests are described in Wisecarver et al., 1985, 19: 211; Bruggemann et al., 1987, J Exp Med 166:1351; Wilkinson et al., 2001, J Immunol Methods 258:183; Patel et al., 1995 J Immunol Methods 184:29. Alternatively, or in addition, ADCC activity of the antibodies of interest can be evaluated in vivo, for example, in an animal model such as the one disclosed in Clynes et al., 1998, PNAS USA 95:652.

“Target cell” herein means a cell expressing a target antigen that can be recognized by an armed NK cell according to the invention or a cell expressing an antibody capable of recognizing an antigen presented by an armed NK cell according to the invention. For example, a target cell can be a cancer cell, a cell infected by a pathogenic agent, an autoreactive B-cell, an autoreactive T-cell.

“Wild-type or WT” means herein an amino acid sequence which is found in nature, including allele variations. A WT protein, a polypeptide, an antibody, an immunoglobulin, an IgG, etc., an amino acid sequence or a nucleotide sequence which has not been intentionally modified.

### Recombinant Polypeptide

The present invention uses a recombinant polypeptide suitable for attaching to an NK cell or an NK cell precursor.

This recombinant polypeptide comprises (i) a modified Fc (fragment crystallizable) region, which improves the binding properties of the recombinant polypeptide with respect to the same polypeptide in wild-type form, and (ii) a ligand binding domain. For example, a polypeptide comprising such a modified Fc region has an increased stability and binding specificity with respect to an Fc receptor, more particularly with respect to the FcγRIII receptor (CD16). Furthermore, a recombinant polypeptide comprising such a modified Fc region allows stabilizing said polypeptide at the cell membrane inhibiting the internalization thereof. Therefore, the recombinant polypeptides of the present invention are optimized versions of the parent non-modified polypeptide, that is, the wild-type polypeptide. More precisely, the recombinant polypeptide comprises (i) an Fc region consisting of the constant heavy chain portion beyond the hinge portion comprising two C_{H}2 and C_{H}3 domains (C_{H}2 domain and/or C_{H}3 domain), allowing attachment to Fc receptors and (ii) a region capable of binding to a ligand, allowing recognition of target cells.

In the context of IgG antibodies, each of the IgG isotypes has three CH regions. Consequently, the “CH” domains in the context of the IgG are as follows: “C_{H}1” refers to positions 118-215 according to EU numbering. “C_{H}2” refers to positions 231-340 according to EU numbering, and “C_{H}3” refers to positions 341-446 according to EU numbering.

According to certain embodiments, a recombinant polypeptide according to the invention has the formula (I):

[Modified Fc region]-[Linker]ₓ-[Ligand binding domain]

wherein:
the [modified Fc region] is selected from the different embodiments of the modified Fc region and/or the modified C_{H}2 domain of the modified Fc region, as defined in the present description,
X is an integer equal to 0 or 1;
[Linker] comprises a polypeptide sequence having 1 to 100 amino acids;
[Ligand binding domain] comprises a sequence capable of binding to a target ligand, such as a target substance, a target compound, a target molecule or even a target cell, (i) for example a ligand binding domain which is capable of binding to a specific antigen of a target cell, or a sequence capable of binding to a natural or tumor antigen, such as the antigen binding domain of an antibody, which is capable of binding to said target or (ii) for example a ligand binding domain which is capable of binding to a cell receptor or binding to the antigen binding domain of an antibody, in which case the ligand binding domain comprises, or consists of, an antigen recognized by the antigen binding domain of said antibodies.

The modified Fc region can be a wild-type amino acid sequence having one or several (for example 1 to 10 or more) amino acid substitutions or deletions with respect to the wild-type amino acid sequence, for example, in the hinge region or C_{H}2 or C_{H}3. Therefore, the amino acid sequence of the Fc region has an amino acid sequence identity of at least about 70%, 75%, 80%, 85%, 90%, 95% or more (that is, 96%, 97%, 98%, 99% or 100%) with the amino acid sequence of the Fc region of the wild-type polypeptide (for example, with reference to a C_{H}2 domain of human IgG1, referred to as “Fc WT” and comprising SEQ ID NO. 1).

In certain embodiments, the modified Fc region comprises an amino acid sequence having at least 90% sequence identity with the amino acid sequence of the Fc region of the wild-type polypeptide (for example, SEQ ID NO. 15 or alternatively SEQ ID NO. 16). The amino acid sequence can further comprise other modifications, for example, reducing the formation of disulfide bonds. In certain embodiments, the modified Fc region and/or the C_{H}2 domain of the modified Fc region comprises the substitution of one or several amino acids with respect to an Fc region of a wild-type human IgG.

In certain embodiments, the modified Fc region comprises an amino acid sequence having at least 95% sequence identity with a C_{H}2 domain of a human IgG, for example, a C_{H}2 domain of a wild-type human IgG1. The C_{H}2 domain can contain other modifications (for example, reducing or eliminating the effector function).

In certain embodiments, the modified Fc region comprises an amino acid sequence having at least 95% sequence identity with a C_{H}3 domain of IgG, for example a C_{H}3 domain of wild-type human IgG1. The C_{H}3 domain can further comprise other modifications for conferring specific allotypes. On the one hand, the C_{H}3 domain can comprise modifications with respect to a different allotype of the wild-type human IgG1. In certain cases, the C_{H}3 domain corresponds to the amino acid sequence of the C_{H}3 domain of wild-type human IgG1s.

In certain embodiments, a modified Fc region comprises at least one amino acid substitution in a position selected from the group consisting of: 230, 233, 234, 235, 236, 239, 240, 243, 264, 266, 268, 272, 274, 275, 276, 278, 302, 318, 324, 325, 326, 327, 328, 329, 330, 331, 332 and 335, where the amino acid numbering in the Fc region is according to EU numbering. In certain preferred embodiments, said modified Fc region comprises an amino acid substitution selected from the group consisting of: P230E, P230Y, P230G, E233N, E233Q, E233K, E233R, E233S, E233T, E233H, E233A, E233V, E233L, E233I, E233F, E233M, E233Y, E233W, E233G, L234K, L234R, L234S, L234A, L234M, L234W, L234P, L234G, L234A, L235E, L235K, L235R, L235A, L235A, L235M, L235W, L235P, L235G, G236D, G236E, G236A, G236N, G236Q, G236K, G236R, G236S, G236T, G236H, G236A, G236V, G236L, G236I, G236F, G236M, G236Y, G236W, G236P, S239Q, S239K, S239R, S239V, S239L, S239I, S239M, S239D, S239W, S239P, S239G, F243E, V264D, V264E, V264N, V264Q, V264K, V264R, V264S, V264H, V264W, V264P, V264G, H268D, H268E, H268Q, H268K, H268R, H268T, H268V, H268L, H268I, H268F, H268M, H268W, H268P, H268G, E272D, E272R, E272T, E272H, E272V, E272L, E272F, E272M, E272W, E272P, E272G, K274D, K274N, K274S, K274H, K274V, K274I, K274F, K274M, K274W, K274P, K274G, F275L, N276D, N276T, N276H, N276V, N276I, N276F, N276M, N276W, N276P, N276G, 278D, Y278N, Y278Q, Y278R, Y278S, Y278H, Y278V, Y278L, Y278I, Y278M, Y278P, Y278G, E318Q, E318H, E318L, E318Y, S324H, S324F, S324M, S324W, S324P, S324G, S324T, N325K, N325R, N325S, N325F, N325M, N325Y, N325W, N325P, N325G, K326P, A327E, A327K, A327R, A327H, A327V, A327I, A327F, A327M, A327Y, A327W, A327P, L328D, L328Q, L328K, L328R, L328S, L328T, L328V, L328I, L328Y, L328W, L328P, L328G, P329D, P329E, P329N, P329Q, P329K, P329G, P329R, P329S, P329T, P329H, P329V, P329L, P329I, P329M, P329Y, P329W, P329G, A330E, A330L, A330N, A330T, A330P, A330G, P331D, P331Q, P331R, P331T, P331L, P331I, P331F, P331M, P331Y, P331W, I332K, I332R, I332S, I332E, I332V, I332L, I332F, I332M, I332W, I332P, I332G, E333L, E333F, E333M, E333P, K334P, T335N, T335S, T335H, T335V, T335L, T335I, T335F, T335M, T335W, T335P, T335G, where the amino acid numbering in the Fc region is according to EU numbering.

In certain embodiments, the modified Fc region and/or the C_{H}2 domain of the modified Fc region comprise amino acid substitution S239D, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitution I332E, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitution S324T, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitution H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitution A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitution G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D and I332E, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D and S324T, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D and H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E and S324T, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E and H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S324T and H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S324T and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S324T and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions H268FL and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions H268F and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions A330L and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E and S324T, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E and H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, S324T and H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, S324T and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, S324T and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, H268F and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, H268F and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, A330L and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E, S324T and H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E, S324T and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E, S324T and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S324T, H268F and S239D, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S324T, H268F and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S324T, H268F and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions H268F, A330L and S239D, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions H268F, A330L and I332E, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions H268F, A330L and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions A330L, G236A and S239D, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions A330L, G236A and I332E, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions A330L, G236A and S324T, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions A330L, G236A and H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E, S324T and H268F, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E, S324T and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E, S324T and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E, H268F and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E, H268F and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S239D, I332E, A330L and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E, S324T, H268F and A330L, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E, S324T, H268F and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions S324T, H268F, A330L and G236A, according to EU numbering, with respect to the Fc WT region, that is, the C_{H}2 domain of a wild-type human IgG1 (for example, SEQ ID NO. 1).

In certain embodiments, the modified Fc region and/or the C_{H}2 domain of the modified Fc region comprises amino acid substitutions I332E, S324T, H268F and A330L, according to EU numbering, with respect to the Fc WT region, that is, the C_{H}2 domain of a wild-type human IgG1 (for example, SEQ ID NO. 1).

In certain embodiments, the variants of the modified Fc region are selected from the group consisting of S239D/H268F/S324T/I332E, S239D, I332E, S239D/I332E, S239D/S324T/I332E, S239D/H268F/I332E, A330L, A330L/I332E, G236A/S239D, G236A/S239D/A330L/I332E, G236A/S239D/I332E, L234A/L235A/P329G, where the numbering of the residues in the Fc region is according to EU numbering.

In certain embodiments, the amino acid sequence of the modified Fc region and/or the C_{H}2 domain of the modified Fc region correspond to the amino acid sequence presented in any one of SEQ ID NO. 2 to 13.

In certain alternative embodiments, the variants of the modified Fc region do not include A330 modification, such as A330L, where the numbering of the residues in the Fc region is according to EU numbering.

Preferably, the [modified Fc region] of the polypeptide of formula (I) is selected from the amino acid sequence presented in any one of SEQ ID NO. 2 to 13.

In a preferred embodiment, the amino acid sequence of the modified Fc region and/or the C_{H}2 domain of the modified Fc region correspond to the amino acid sequence presented in SEQ ID NO. 2, referred to as Fc SDH region.

In certain embodiments, the recombinant polypeptide according to the invention comprises a modified Fc region, wherein the C_{H}2 domain contains or does not contain other modifications.

In certain embodiments, the recombinant polypeptide according to the invention comprises a modified Fc region, wherein the C_{H}3 domain includes or does not include modifications.

In certain embodiments, the recombinant polypeptide according to the invention comprises a modified Fc region, wherein the hinge region can be a hinge region of wild-type IgG1 with or without substitutions.

In certain embodiments, the recombinant polypeptide according to the invention comprises a modified Fc region, wherein (a) the C_{H}2 domain comprises one or several modifications with respect to the C_{H}2 domain of wild-type human IgG1, or (b) the C_{H}3 domain comprises one or several modifications with respect to the C_{H}3 domain of wild-type human IgG1 or (c) the two C_{H}2 and C_{H}3 domains comprise one or several modifications with respect to the C_{H}2 and C_{H}3 domain of wild-type human IgG1, respectively.

In certain embodiments of a recombinant polypeptide according to the invention, the ligand binding domain comprised therein contains, including, but not limited to, the proteins, sub-units, domains, motifs and/or epitopes belonging to the following list of targets: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-ADN, BTC, complement factor 3 (C3), C3a, C4, C5, C5a, C10, CA125, CAD-8, Calcitonin, cAMP, Carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMVUL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-1 (brain IGF-1), Dhh, digoxin, DNAM-1, DNase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, AND-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, FIt-3, FIt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMWMAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INFbeta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muci), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGFbeta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory (Ligand TRANCE/RANK ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGAND HVEM LIGHT, LTg), TNFSF15 (TL1A/VEGI), TNFSF18 (GITR Ligand AITR Ligand, TL6), TNFSFIA (TNF-α Conectine, DIF, TNFSF2), TNFSFIB (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1 BB Ligand CD137 Ligand), TP-1, t-PA, Tpo, TRAIL, TRAIL R, TRAIL-R1, TRAIL-R2, TRANCE, transfert receptor, TRF, Trk, TROP-2, TSG, TSLP, CA 125 tumor-associated antigen, tumor-associated antigen expressing Lewis Y-related carbohydrate, TWEAK, TXB2, Ung, uPAR, uPAR-1, Urokinase, VCAM, VCAM-1, VECAD, VE-Cadherin, VEcadherin-2, VEFGR-1 (fit-1), VEGF, VEGFR, VEGFR-3 (fit-4), VEGI, VIM, Viral antigens, VLA, VLA-1, VLA-4, VNR integrin, von Willebrand factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and hormone and growth factor receptors, etc.

In certain embodiments of a recombinant polypeptide according to the invention, the ligand binding domain comprised therein is capable of binding to a ligand defined by, but not limited to, the proteins, sub-units, domains, motifs and/or epitopes belonging to the following list of targets: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-ADN, BTC, complement factor 3 (C3), C3a, C4, C5, C5a, C10, CA125, CAD-8, Calcitonin, cAMP, Carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMVUL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-1 (brain IGF-1), Dhh, digoxin, DNAM-1, DNase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, AND-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, FIt-3, FIt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMWMAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, I-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INFbeta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muci), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGFbeta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, 5100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta RII, TGF-beta Rllb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-R1, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1 BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (Ligand Apo-2 TRAIL, TL2), TNFSF11 (Ligand TRANCE/RANK ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGAND HVEM LIGHT, LTg), TNFSF15 (TL1A/VEGI), TNFSF18 (GITR Ligand AITR Ligand, TL6), TNFSF1A (TNF-a Conectine, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1 BB Ligand CD137 Ligand), TP-1, t-PA, Tpo, TRAIL, TRAIL R, TRAIL-R1, TRAIL-R2, TRANCE, transfert receptor, TRF, Trk, TROP-2, TSG, TSLP, CA 125 tumor-associated antigen, tumor-associated antigen expressing Lewis Y-related carbohydrate, TWEAK, TXB2, Ung, uPAR, uPAR-1, Urokinase, VCAM, VCAM-1, VECAD, VE-Cadherin, VEcadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and hormone and growth factor receptors, etc.

According to a preferred embodiment of the recombinant polypeptide, the ligand binding domain is capable of binding to a target molecule such as an antibody. According to this embodiment, the ligand binding domain is suitable for binding to an antibody, and most particularly to the variable region of an antibody.

According to one embodiment, a recombinant polypeptide of the invention, that is a polypeptide having (i) a modified Fc (fragment crystallizable) region, and (ii) a ligand binding domain which is capable of binding to a ligand, which is not an antibody.

Particularly, according to one embodiment, a recombinant polypeptide of the invention, that is a polypeptide having (i) a modified Fc region, and (ii) a ligand binding domain, does not have a variable or hypervariable region of an antibody.

According to one embodiment, a recombinant polypeptide of the invention, that is a polypeptide having (i) a modified Fc region, and (ii) a ligand binding domain, said Fc region being capable of binding to said NK cell or precursor thereof, can be an antibody.

In one embodiment, a recombinant polypeptide of the invention can be a completely human antibody or a humanized antibody, and in addition, compared to the same antibody which does not have a modified Fc region, it can have one or several improved or modified properties. These properties comprise the binding specificity with respect to the FcγRIII receptor of the NK cells or any cell expressing this FcγRIII receptor or the improvement of the antibody-dependent cellular cytotoxicity.

In certain embodiments when a recombinant polypeptide according to the invention is an antibody, or fragment thereof, it can be a starting antibody having a known structure and the Fc portion of which has been modified as defined in the present description. The starting antibody having a known structure can be selected from Abagovomab, Abatacept, Abciximab, Abituzumab, Abrilumab, Actoxumab, Adalimumab, Adecatumab, Aducanumab, Aflibercept, Afutuzymab, Alacizumab, Alefacept, Alemtuzumab, Alirocumab, Altumomab, Amatixumab, Anatumomab, Anetumab, Anifromumab, Anrukinzumab, Apolizumab, Arcitumomab, Ascrinvacumab, Aselizumab, Atezolizumab, Atinumab, Altizumab, Atorolimumab, Bapineuzumab, Basiliximab, Bavituximab, Bectumomab, Begelomab, Belatacept, Belimumab, Benralizumab, Bertilimumab, Besilesomab, Bevacizumab, Bezlotoxumab, Biciromab, Bimagrumab, Bimekizumab, Bivatuzumab, Blinatumomab, Blosozumab, Bococizumab, Brentuximab, Briakimumab, Brodalumab, Brolucizumab, Bronticizumab, Canakinumab, Cantuzumab, Caplacizumab, Capromab, Carlumab, Catumaxomab, Cedelizumab, Certolizumab, Cetixumab, Citatuzumab, Cixutumumab, Clazakizumab, Clenoliximab, Clivatuzumab, Codrituzumab, Coltuximab, Conatumumab, Concizumab, Crenezumab, Dacetuzumab, Daclizumab, Dalotuzumab, Dapirolizumab, Daratumumab, Dectrekumab, Demcizumab, Denintuzumab, Denosumab, Derlotixumab, Detumomab, Dinutuximab, Diridavumab, Dorlinomab, Drozitumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab, Enlimomab, Enoblituzumab, Enokizumab, Enoticumab, Ensituximab, Epitumomab, Epratuzomab, Erlizumab, Ertumaxomab, Etanercept, Etaracizumab, Etrolizumab, Evinacumab, Evolocumab, Exbivirumab, Fanolesomab, Faralimomab, Farletuzomab, Fasimumab, Felvizumab, Fezkimumab, Ficlatuzumab, Figitumumab, Firivumab, Flanvotumab, Fletikumab, Fontolizumab, Foralumab, Foravirumab, Fresolimumab, Fulramumab, Futuximab, Galiximab, Ganitumab, Gantenerumab, Gavilimomab, Gemtuzumab, Gevokizumab, Girentuximab, Glembatumumab, Golimumab, Gomiliximab, Guselkumab, Ibalizumab, Ibritumomab, Icrucumab, Idarucizumab, Igovomab, Imalumab, Imciromab, Imgatuzumab, Inclacumab, Indatuximab, Indusatumab, Infliximab, Intetumumab, Inolimomab, Inotuzumab, Ipilimumab, Iratumumab, Isatuximab, Itolizumab, Ixekizumab, Keliximab, Labetuzumab, Lambrolizumab, Lampalizumab, Lebrikizumab, Lemalesomab, Lenzilumab, Lerdelimumab, Lexatumumab, Libivirumab, Lifastuzumab, Ligelizumab, Lilotomab, Lintuzumab, Lirilumab, Lodelcizumab, Lokivetmab, Lorvotuzumab, Lucatumumab, Lulizumab, Lumiliximab, Lumretuzumab, Mapatumumab, Margetuximab, Maslimomab, Mavrilimumab, Matuzumab, Mepolizumab, Metelimumab, Milatuzumab, Minetumomab, Mirvetuximab, Mitumomab, Mogamulizumab, Morolimumab, Motavizumab, Moxetumomab, Muromonab-CD3, Nacolomab, Namilumab, Naptumomab, Narnatumab, Natalizumab, Nebacumab, Necitumumab, Nemolizumab, Nerelimomab, Nesvacumab, Nimotuzumab, Nivolumab, Nofetumomab, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab, Oregovomab, Orticumab, Otelixizumab, Oltertuzumab, Oxelumab, Ozanezumab, Ozoralizumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab, Pintumomab, Polatuzumab, Ponezumab, Priliximab, Pritumumab, Quilizumab, Racotumomab, Radretumab, Rafivirumab, Ralpancizumab, Ramucirumab, Ranibizumab, Raxibacumab, Refanezumab, Regavirumab, Reslizumab, Rilonacept, Rilotumumab, Rinucumab, Rituximab, Robatumumab, Roledumab, Romosozumab, Rontalizumab, Rovelizumab, Ruplizumab, Sacituzumab, Samalizumab, Sarilumab, Satumomab, Secukimumab, Seribantumab, Setoxaximab, Sevirumab, Sibrotuzumab, Sifalimumab, Siltuximab, Siplizumab, Sirukumab, Sofituzumab, Solanezumab, Solitomab, Sonepcizumab, Sontuzumab, Stamulumab, Sulesomab, Suvizumab, Tabalumab, Tacatuzumab, Tadocizumab, Talizumab, Tanezumab, Taplitumomab, Tarextumab, Tefibazumab, Telimomab aritox, Tenatumomab, Teneliximab, Teplizumab, Tesidolumab, TGN 1412, Ticlimumab, Tildrakizumab, Tigatuzumab, TNX-650, Tocilizumab, Toralizumab, Tosatoxumab, Tositumomab, Tovetumab, Tralokimumab, Trastuzumab, TRBS07, Tregalizumab, Tremelimumab, Trevogrumab, Tucotuzumab, Tuvirumab, Ublituximab, Ulocuplumab, Urelumab, Urtoxazumab, Ustekimumab, Vandortuzumab, Vantictumab, Vanucizumab, Vapaliximab, Varlimumab, Vatelizumab, Vedolizumab, Veltuzumab, Vepalimomab, Vesencumab, Visilizumab, Volocixumab, Vorsetuzumab, Votumumab, Zalutumimab, Zanolimumab, Zatuximab, Ziralimumab, Ziv-Aflibercept, and Zolimomab.

### NK (Natural Killer) Cells

In one of these objects, the present invention uses an NK (Natural Killer) cell, preferably allogeneic or autologous with respect to an individual in need thereof, or an NK cell precursor.

An NK cell is a lymphocyte capable of spontaneously destroying target cells, involving MHC class 1 molecules. The specificity of these NK cells is to be capable of lysing diseased cells without requiring prior activation and without contacting the pathogenic agent.

The NK cells of the present invention can be derived from any source comprising such cells. The NK cells are found in many tissues and can be obtained, for example, from the lymph nodes, spleen, liver, lungs, intestines, deciduas and can also be obtained from iPS cells or from embryonic stem cells (ESC). Generally, cord blood, peripheral blood, mobilized peripheral blood and bone marrow, which contain heterogeneous lymphocytic cell populations, are used to provide a large number of NK cells for research and clinical use.

Therefore, according to certain embodiments of the present invention, the method comprises culturing an NK cell population, preferably allogeneic or autologous with respect to an individual in need thereof, derived from one of the following elements: cord blood, peripheral blood or bone marrow and preferably cord blood. In certain embodiments, the NK cells are cultured from a heterogeneous cell population comprising NK cells, CD3⁻ cells and CD3⁺ cells. In another variant, the NK cell population is screened or enriched with respect to the NK cells. In certain cases, the NK cells can be propagated from fresh cell populations, whereas other cases propagate NK cells from stocked cell populations (such as cryopreserved and thawed cells) or from previously cultured cell populations.

The NK cells are associated with the fraction of mononuclear cells from cord blood or peripheral blood or bone marrow. In one embodiment, the cell population comprising said NK cells is a mononuclear cell population or a total nuclear cell population depleted of CD3⁺ cells or of CD3⁺/CD19⁺ cells.

In another embodiment, the cell population comprising NK cells is a non-screened NK cell population. In another embodiment, the cells are screened and the NK cells comprise CD45⁺/CD56⁺/CD3⁻ and/or CD45⁺/CD56⁺/CD3⁻/CD16⁺ cells. Methods of screening for NK cells based on phenotype (for example, immunodetection and analysis by flow cytometry) are well known to those skilled in the art.

Most frequently, the cord blood or bone marrow samples are then treated to obtain cell populations before placing the NK cells in the culture medium (or buffer). For example, the cord blood sample can be treated to enrich or purify or isolate specific defined cell populations. The terms “purify” and “isolate” do not require absolute purity; rather they are relative terms. Therefore, for example, a purified NK cell population is a population wherein the specified cells are more enriched than those found in their original tissue. A substantially pure NK cell preparation can be enriched such that the desired cells represent at least 50% of the total cells present in the preparation. In certain types of preparations, a substantially pure NK cell population represents at least 60%, 70%, 80%, 85%, 90% or 95% or more of the total cells present in the preparation.

Methods of enriching and isolating lymphocytes (CD45⁺ cells) are well known in the field. For example, according to one embodiment, the red blood cells are removed from the biological sample in order to keep only the lymphocytes. In its simplest form, the removal of red blood cells can imply the centrifugation of non-coagulated whole blood or bone marrow. Based on density, the red blood cells are separated from the lymphocytes and other cells. Fractions rich in lymphocytes can then be selectively recovered. The lymphocytes and progenitors thereof can also be enriched by centrifugation using separation media such as standard lymphocytes separation medium (LSM) available from several commercial sources. The lymphocytes and progenitors thereof can also be enriched using several procedures based on affinity. Many affinity separation methods based on antibodies are known in the art, such as magnetic beads conjugated to antibodies. The lymphocytes can also be enriched by means of preparations available on the market for the negative selection of undesired cells, such as FICOLL-HYPAQUE™ and other density gradient media formulated for enrichment of NK cells.

Methods for selecting NK cells from blood, bone marrow or tissue samples are well known in the field (see, for example, U.S. Pat. No. 5,770,387 by Litwin et al.). The most commonly used protocols are based on the isolation and the purification of CD56⁺ cells, generally after fractioning mononuclear cells, and the depletion of non-NK cells such as CD3⁺, CD34⁺, CD133⁺, etc. Combinations of two or more protocols can be used to provide NK cell populations having a greater purity with respect to non-NK contaminants. Kits available on the market for isolating NK cells comprise one-step procedures (for example, CD56 microbeads and kits for isolating CD56⁺, CD56⁺/CD16⁺ from Miltenyi Biotec, Auburn CA), and procedures in several steps, including depletion, or partial depletion, of CD3⁺ or depletion with non-NK cell antibodies recognizing and removing T-cells (for example, OKT-3), B-cells, stem cells, dendritic cells, monocytes, granulocytes and erythroid cells. Therefore, in certain implementations, the NK cells are screened for CD45⁺/CD56⁺CD3⁻, CD45⁺/CD56⁺/CD3⁻/CD16⁺, CD45⁺/CD56⁺/CD3⁻/CD16⁻, preferably CD45⁺/CD56⁺/CD3⁻/CD16⁺.

The NK cells are then cultured and kept by any method known to those skilled in the art, before being incubated with the recombinant polypeptides of the invention, obtaining armed NK cells. The NK cells can be kept for up to 7 days, or up to 3 weeks before being “armed”. Therefore, in certain embodiments, the NK cell population is cultured for at least 3, at least 5, at least 7, potentially 10, potentially 12, potentially 14, potentially 16, potentially 18, potentially 20 and potentially 21 days, or 1, 2 or 3 weeks, 4 weeks, 5 weeks, 6 weeks, or more.

The NK cell populations can be cultured by means of several methods and devices. The choice of culturing apparatus is generally based on the scale and aim of the culture. The scaling of the cell culture preferably implies the use of dedicated apparatus. The apparatus for the production of NK cells on a large scale and of clinical quality are detailed, for example, in Spanholtz et al. (PLoS ONE 2010; 5: e9221) and Sutlu et al. (Cytotherapy 2010, Early Online 1-12).

In a certain embodiment, the NK cells used in the invention are cells from a human other than the individual to be treated, referred to as allogeneic.

In a certain embodiment, the NK cells used in the invention are cells from the individual himself or herself to be treated, referred to as autologous.

NK Cell in Combination with a Recombinant Polypeptide

According to one of these objects, the present application relates to an NK cell allogeneic or autologous with respect to an individual in need thereof, or an NK cell precursor attached to a recombinant polypeptide, wherein said recombinant polypeptide comprises (i) a modified Fc region, capable of binding to said NK cell or precursor thereof, and (ii) a domain capable of binding to an antigen; for use as a medicament.

Two embodiments of a recombinant polypeptide according to the invention are illustrated in FIG. 8. A recombinant polypeptide according to the invention is represented on the left-hand side of FIG. 8, comprising (i) a modified Fc region and (ii) a ligand binding domain, wherein the ligand binding domain comprises, or consists of, an antigen. A recombinant polypeptide according to the invention is represented on the right-hand side of FIG. 8, comprising (i) a modified Fc region and (ii) a ligand binding domain, wherein the ligand binding domain comprises, or consists of, the antigen binding domain of an antibody. Therefore, the inventors have implemented a universal cell therapy product based on NK cells. In contrast to CAR-T cells, the specificity to destroy the target cells is not obtained by the NK cells themselves but by specific peptides recognizing these target cells. The present combination, NK cell—recombinant polypeptide, can therefore be extended to all kinds of pathologies, by designing specific polypeptides, specifically combining a specific antigen to the recombinant polypeptide of the invention.

The present invention provides compounds of construct (A) such as:

[NK cell]-[recombinant polypeptide of formula (I)]

The present invention encompasses the compounds of construct (B):

[NK cell]-[[Modified Fc region]-[Linker]ₓ-[Ligand binding domain]]
wherein [NK cell] is an NK cell as previously defined in the present application, [recombinant polypeptide of formula (I)] is a polypeptide or an antibody as previously defined in the present application.

In one embodiment, the ligand binding domain is capable of binding to an antigen-type ligand, which antigen can be selected from viral antigens, tumor antigens, infectious disease antigens, autoimmune antigens, toxins or combinations thereof.

In certain embodiments, the ligand binding domain is capable of binding to a ligand which is a peptide concatemer. In certain embodiments, the ligand binding domain is capable of binding to a ligand selected from glutamate decarboxylase 65 (GAD 65), natural DNA, myelin basic protein, myelin proteolipid protein, acetylcholine receptor component, thyroglobulin, thyroid-stimulating hormone (TSH) receptor and citrullinated peptides. In certain other embodiments, the ligand binding domain is capable of binding to an antigen-type ligand, which antigen is selected from a cancer antigen, infectious disease antigen selected from bacteria, viral, parasite and fungal antigens, autoimmune disease antigen and the derivative thereof.

According to one embodiment, a cancer antigen, to which the ligand binding domain of a recombinant polypeptide according to the invention is capable of binding, can be an antigen of a cancer selected from: melanoma, kidney cancer, prostate cancer, breast cancer, colon cancer, ovarian cancer, lung cancer, bone cancer, pancreatic cancer, skin cancer, head or neck cancer, skin or intraocular malign melanoma, uterine cancer, rectal cancer, anal cancer, stomach cancer, testicular cancer, uterine cancer, Fallopian tube carcinoma, endometrial carcinoma, cervical carcinoma, vaginal carcinoma, vulvar carcinoma, Hodgkin's disease, non-Hodgkin's lymphoma, esophageal cancer, small intestine cancer, endocrine system cancer, thyroid cancer, parathyroid cancer, adrenal gland cancer, soft tissue sarcoma, urethral cancer, penile cancer, chronic or acute leukemias including acute myeloid leukemia, chronic myeloid leukemia, acute lymphoblastic leukemia, chronic lymphocytic leukemia, childhood solid tumors, lymphoid lymphoma, bladder cancer, kidney or urethral cancer, renal pelvis carcinoma, central nervous system (CNS) neoplasm, CNS primary lymphoma, tumor angiogenesis, spinal tumor, brainstem glioma, pituitary adenoma, Kaposi's sarcoma, epidermoid cancer, squamous cell cancer, T-lymphocyte lymphoma, environmentally-induced cancers and combinations of said cancers.

According to certain embodiments, an infectious disease antigen, to which the ligand binding domain of a recombinant polypeptide according to the invention is capable of binding, can be an infectious disease antigen selected from: (a) a disease selected from flu, herpes, giardiasis, malaria and leishmaniasis; (b) a pathogen infection with a virus selected from human immunodeficiency virus (HIV), hepatitis virus, herpes virus, adenovirus, influenza virus, flavivirus, echovirus, rhinovirus, coxsackievirus, cornovirus, respiratory syncytial virus, mumps virus, rotavirus, measles virus, rubella virus, parvovirus, vaccinia virus, HTLV virus, dengue virus, papillomavirus, molluscum virus, poliomyelitis virus, rabies virus, JC virus and arboviral encephalitis virus; (c) a pathogen infection with a bacterium selected from Chlamydia, rickettsia bacteria, mycobacteria, staphylococcus, streptococcus, pneumococcus, meningococcus and gonococcus, Klebsiella, Proteus, Serratia, Pseudomonas, Legionella, Diphtheria, Salmonella, Bacilli, cholera, tetanus, botulism, carbon disease, plague, leptospirosis and Lyme disease bacteria; (d) a pathogen infection with a fungus selected from Candida, Cryptococcus neoformans, Aspergillus, Genus Mucorales, Sporothrix schenka, Blastomyces dermatitidis, Paracoccidioides brasiliensis, Coccidioides immitis and Histoplasma capsulatum; or (e) a pathogen infection with a parasite selected from Entamoeba histolytica, Balantidium coli, Naegleria fowleri, Acanthamoeba sp., Giardia lamblia, Cryptosporidium sp., Pneumocystis carina, Plasmodium virax, Babesia microti, Trypanosoma brucei, Trypanosoma cruzi, Leishmania donovani, Toxoplasma gondii and Nippostrongylus brasiliensis.

According to certain embodiments, an autoimmune disease antigen or a derivative thereof, to which the ligand binding domain of a recombinant polypeptide according to the invention is capable of binding, can be an antigen selected from the following diseases or derivatives: organ graft rejection, graft-versus-host disease, rheumatoid arthritis, systemic lupus erythematosus, scleroderma, primitive Sjôgren's syndrome (or Gougerot-Sjôgren's syndrome), autoimmune polyneuropathies such as multiple sclerosis, type-I diabetes, autoimmune hepatitis, ankylosing spondylitis, Reiter's syndrome, gouty arthritis, celiac disease, Crohn's disease, Hashimoto's thyroiditis, Addison's disease, autoimmune hepatitis, Basedow's disease, ulcerative colitis, vasculitis such as ANCA (antineutrophil cytoplasmic antibodies)-associated systemic vasculitis, autoimmune cytopenias and other hematologic complications in adults and children, such as acute or chronic autoimmune thrombopenia, autoimmune hemolytic anemias, hemolytic disease of the newborn (HDN), cold agglutinin disease, thrombotic thrombocytopenic purpura and autoimmune acquired hemophilia, Goodpasture syndrome, extramembranous nephropathy, autoimmune oily skin conditions, refractory myasthenia, mixed cryoglobulinemias, psoriasis, juvenile idiopathic arthritis, inflammatory myositis, dermatomyositis and systemic autoimmune conditions in children including antiphospholipid syndrome.

According to certain embodiments, the antigen, to which the ligand binding domain of a recombinant polypeptide according to the invention is capable of binding, can be an antigen selected from the following diseases or derivatives: thrombocythemia, myelodysplastic syndromes, benign tumors and for senescent cell removal.

In certain embodiments, the armed NK cells of the present invention are used for lysing the target cells carrying the antigen that can be recognized by the recombinant polypeptide of the invention, for example cancer cells.

In certain embodiments, the armed NK cells of the present invention are used for lysing the target cells which recognize the antigen attached to the recombinant polypeptide of the invention, for example the autoreactive B-cells.

According to certain embodiments, the armed NK cells can be kept for up to 7 days, or up to 3 weeks before being injected into an individual in need thereof. Therefore, in certain embodiments, the armed NK cell population is cultured for at least 3, at least 5, at least 7, potentially 10, potentially 12, potentially 14, potentially 16, potentially 18, potentially 20 and potentially 21 days, or 1, 2 or 3 weeks, 4 weeks, 5 weeks, 6 weeks, or more. The exemplary and non-limiting culture durations, as detailed in the examples of the application, are for at least 3 days and at least 7 days.

### Pharmaceutical Composition

Compositions are also provided, specifically pharmaceutical compositions, comprising an NK (Natural Killer) cell or an NK cell precursor, in combination with a recombinant polypeptide; wherein said recombinant polypeptide comprises (i) a modified Fc (fragment crystallizable) region, and (ii) a ligand binding domain, said Fc region being capable of binding to said NK cell or precursor thereof, formulated with a pharmacologically or pharmaceutically acceptable excipient or a vehicle. These compositions can comprise one or several combinations of NK cells armed with recombinant polypeptides according to the invention (for example, two or more different ones). For example, a pharmaceutical composition described herein can comprise a combination of armed NK cells which bind to different epitopes on the target antigen or antibodies having complementary activities (or immunoconjugates or bispecific compounds).

In certain cases, the composition comprises at least 1 mg/ml, 5 mg/ml, 10 mg/ml, 50 mg/ml, 100 mg/ml, 150 mg/ml, 200 mg/ml, 1-300 mg/ml or 100-300 mg/ml of armed NK cells.

The pharmaceutical compositions described herein can also be administered in combination therapy, that is, in combination with other agents. For example, the combination therapy can include an armed NK cell described herein in combination with at least another antipathogenic agent and/or a stimulating agent for the NK cells (for example, an activator). Examples of therapeutic agents which can be used in combination are described in more detail hereinafter in the section concerning the use of the armed NK cells described in the present application.

In certain cases, the pharmaceutical compositions described herein can include other compounds, agents and/or medicaments used for treating pathologies such as a cancer, an autoimmune disease and derivatives thereof or an infectious disease. These compounds, agents and/or medicaments can include, for example, chemotherapeutic agents, small molecule agents or antibodies stimulating an immune response against a given cancer.

The pharmaceutical compositions described herein can comprise one or several pharmaceutically acceptable salts. “Pharmaceutically acceptable salt” means a salt conserving the desired biological activity of the parent compound and not transmitting undesirable toxic effects. Acid addition salts and base addition salts are examples of salts. The acid addition salts comprise non-toxic inorganic acids such as hydrochloric acid, nitric acid, phosphoric acid, sulfuric acid, hydrobromic acid, hydroiodic acid, phosphoric acid and mono- and di-carboxylic aliphatic acids, phenyl-substituted alkanoic acids, hydroxyalkanes. Salts derived from non-toxic organic acids such as acids, aromatic acids, aromatic and aliphatic sulfonic acids are included. The base addition salts comprise, for example, alkaline earth metals such as sodium, potassium, magnesium and calcium, as well as N,N′-dibenzylethylenediamine, N-methylglucamine, chloroprocaine, choline, diethanolamine, ethylenediamine, procaine and other. Salts derived from toxic organic amines are included.

The pharmaceutical compositions described herein can also include a pharmaceutically acceptable antioxidant. Examples of pharmaceutically acceptable antioxidants comprise (1) water-soluble antioxidants such as ascorbic acid, cysteine hydrochloride, sodium bisulfate, sodium metabisulfite, sodium sulfite, etc., (2) oil-soluble antioxidants, oxidizing agents such as ascorbyl palmitate, butylated hydroxyanisole (BHA), butylated hydroxytoluene (BHT), lecithin, propyl gallate, alpha-tocopherol and others; and (3) metal chelators such as citric acid, ethylenediaminetetraacetic acid (EDTA), sorbitol, tartaric acid, phosphoric acid and others are included.

Examples of suitable aqueous or non-aqueous carriers that can be used in the pharmaceutical compositions described herein comprise water, ethanol, polyols (for example, glycerol, propylene glycol, polyethylene glycol, etc.) and suitable mixtures thereof, vegetable oils such as olive oil and injectable organic esters such as ethyl oleate. Suitable flowability can be maintained, for example, conserving the required particle size in the case of a dispersion and using surfactants. These compositions can also contain adjuvants such as preservatives, wetting agents, emulsifying agents and dispersing agents. The prevention of the presence of microorganisms can be ensured by both the sterilization methods described hereinafter and the inclusion of several antibacterial and antifungal agents such as parabens, chlorobutanol, phenol, sorbic acid and others. It can also be desirable to include isotonic agents, such as sugars, sodium chloride, in the composition. Furthermore, the inclusion of absorption-retardant agents, such as aluminium monostearate and gelatin, can delay the absorption of injectable pharmaceutical forms.

Pharmaceutically acceptable carriers comprise sterile aqueous solutions or dispersions and sterile powders for the extemporary preparation of sterile injectable solutions or dispersions. The use of such carriers and agents for pharmaceutically active substances is well known in the field. Except where a conventional medium or agent is not compatible with the active ingredient, the use thereof in the pharmaceutical compositions described herein is contemplated. Complementary active substances can also be incorporated in the compositions.

A pharmaceutical composition must generally be sterile and stable under manufacturing and storage conditions. The composition can be formulated in the form of a solution, microemulsion, solution, microemulsion, liposome or other ordered structure suited for high medicament concentration. The carrier can be a solvent or a dispersion medium containing, for example, water, ethanol, polyol (for example, liquid glycerol, propylene glycol and polyethylene glycol, and others), and suitable mixtures thereof. Suitable flowability can be maintained, for example, by use of a coating such as lecithin, by conservation of the required particle size in the case of a dispersion and by the use of surfactants. In many cases, it will be preferable to include isotonic agents, for example sugars, polyalcohols such as mannitol and sorbitol, or sodium chloride in the composition. Delayed absorption of the injectable compositions can be brought about by the inclusion of an absorption-retardant agent, for example, monostearate salts and gelatin, in the composition.

The sterile injectable solutions can be prepared including the active compound, that is, armed NK cells, in a required amount in a suitable solvent, potentially with one or a combination of ingredients listed hereinafter, then sterilizing by means of microfiltration. Generally, the dispersions are prepared incorporating the active compound in a sterile vehicle containing a basic dispersion medium and other necessary ingredients from those listed hereinafter. In the case of sterile powders for the preparation of sterile injectable solutions, the preferred preparation method is a method of drying under vacuum, wherein an active ingredient powder plus any other desired ingredient is prepared from the previously sterilized and filtered solution, and lyophilizing.

The amount of active ingredient which can be combined with carrier materials to prepare a unit dosage form can vary according to the subject to be treated and the particular mode of administration. The amount of active ingredient, that is, armed NK cells, which can be combined with a carrier material to prepare a unit dosage form, will generally be the amount of the composition which causes a therapeutic effect. According to certain embodiments, the amount of active ingredient, that is, armed NK cells, is about 0.01% to about 99% with respect to the final composition amount, preferably about 0.1% to about 70% with respect to the final composition amount, most of the time in combination with a pharmaceutically acceptable carrier.

Dosage schedules are adjusted in order to obtain the desired optimal response (for example, a therapeutic response). According to certain embodiments, a single bolus administration is possible and several divided doses can be administered over a long period of time, or the dose can be proportionally reduced or increased as indicated in a situation of imminent treatment. Preferably, the formulation of parenteral compositions is in the form of unit doses, specifically in order to facilitate administration and dosage uniformity.

According to certain embodiments, a unit dosage form designates a physical unit which is convenient as a unit dose for the individual to be treated; each unit, associated with the required pharmaceutical carrier, causes the desired therapeutic effect.

According to certain embodiments, for the administration of armed NK cells in combination with or without an antigen, the dosage ranges between about 0.0001 and 100 mg, more generally between 0.01 and 5 mg per kg of body weight of the host. For example, the dosage is comprised between 0.3 mg/kg of body weight, 1 mg/kg of body weight, 3 mg/kg of body weight, 5 mg/kg of body weight or 10 mg/kg of body weight, or 1-10 mg/kg.

According to certain embodiments, a treatment regimen can be one administration once a week, once every two weeks, once every three weeks, once every four weeks, once a month, once every three months or once every three months to six months.

According to certain embodiments, the preferred dosage schedules for the administration of the pharmaceutical compositions according to the invention comprise 1 mg/kg of body weight or 3 mg/kg of body weight per intravenous administration. Specifically, the composition is administered (i) once every 4 weeks, followed by (ii) once every 3 months.

In certain embodiments, two or more armed NK cells having different binding specificities are administered simultaneously, in which case the dose of each armed NK cell administered is within the indicated ranges. The armed NK cell is generally administered several times. The interval between the unit doses can be, for example, once a week, once a month, every three months or once a year. In addition, the intervals can be irregular. In certain embodiments, the dosage is adjusted in order to obtain an armed NK cell plasma concentration of about 1-1000 μg/ml, and in certain embodiments, an armed NK cell plasma concentration of about 25-300 μg/ml.

The armed NK cell can be administered in the form of a sustained release formulation, in which case less frequent administration is required. Dosage and frequency vary depending on the armed NK cell half-life in the patient. The armed NK cells of the invention have a half-life of at most about 3 days and up to about 7 days in vivo.

According to certain embodiments, the pharmaceutical composition is used for a prophylactic or therapeutic treatment. Dosage and frequency of administration can vary based on whether the treatment is prophylactic or therapeutic. In the prophylactic applications, relatively weak doses are administered over long periods at relatively infrequent intervals. Certain patients continue to receive treatment for the rest of their life. In therapeutic applications, relatively high doses at relatively short intervals can be required until progression of the disease is reduced or stopped, preferably until the patient shows a partial or complete improvement of the symptoms of the disease. Thereafter, the patient can be administered a prophylactic regimen.

The real dosage levels of the active ingredients of the pharmaceutical compositions according to the invention are not toxic for the patient in order to obtain the desired therapeutic response for the individual, particularly the composition and the mode of administration. It is possible to vary same in order to obtain an effective amount of active ingredient. The selected dosage level depends on the particular composition being used, or the activity of its ester, salt or amide, the route of administration, the time of administration, the elimination rate of the particular compound being used, the duration of treatment, other medicaments, compounds and/or substances used in combination with a particular composition, the age, sex, weight, condition, general health and medical history of the individual to be treated, as well as similar factors well known in the medical field. The dosage level can also vary depending on several pharmacokinetic factors.

According to certain embodiments, a therapeutically effective dose can prevent or delay the onset of a pathology. For example, laboratory tests used to diagnose a disease comprise chemistry, hematology, serology and radiology. Consequently, the clinical or biochemical assays which monitor one of the elements hereinabove can be used to determine if a particular treatment is a therapeutically effective dose for treating the disease. One skilled in the art can determine such amounts depending on factors such as the size of the individual, the severity of the symptoms of the individual, and the particular composition or the route of administration selected.

The pharmaceutical compositions according to the invention can be administered according to one or several methods known in the art, by one or several routes of administration. As will be appreciated by those skilled in the art, the route of administration and/or the mode of administration will vary depending on the desired result.

According to certain preferred embodiments, the routes of administration for the armed NK cells according to the invention comprise the administration by intravenous, intramuscular, intradermal, intraperitoneal, subcutaneous, spinal route or any other parenteral route of administration such as injection or perfusion.

The armed NK cells according to the invention can also be administered by a topical, epithelial or mucosal route of administration, for example a non-parenteral route, such as intranasal, oral, vaginal, rectal, sublingual or topical.

The active compounds can be prepared with carriers that will protect the compound against a fast release, such as a controlled release formulation, including implants, transdermal patches and microencapsulated delivery systems. Biodegradable and biocompatible polymers can be used, such as ethylene-vinyl acetate, polyanhydrides, polyglycolic acid, collagen, polyorthoesters and polylactic acid. Many methods for preparing these formulations are patented or generally known to the skilled persons (see, for example, Sustained and Controlled Release Drug Delivery Systems, J. R. Robinson, ed., Marcel Dekker, Inc, New York, 1978).

The therapeutic composition can be administered by means of medical devices known in the field. According to certain particular embodiments of the invention, the therapeutic compositions according to the invention include the devices described in U.S. Pat. Nos. 5,399,163; 5,383,851; 5,312,335; 064,413; 4,941,880; 4,790,824; or a hypodermic injection device without a needle such as the device described in U.S. Pat. No. 4,596,556.

Among the examples of implants and modules well known used with the armed NK cells according to the invention, the following can be mentioned: U.S. Pat. No. 4,487,603 which discloses an implantable microperfusion pump for the administration of medicaments at a controlled flow rate; U.S. Pat. No. 4,486,194 which discloses a therapeutic device for administering a medicament through the skin; U.S. Pat. No. 4,447,233 which discloses a perfusion pump for administering a medicament at an accurate perfusion rate; U.S. Pat. No. 4,447,224 which discloses a variable flow rate implantable perfusion device for the continuous administration of a medicament; U.S. Pat. No. 4,439,196 which discloses an osmotic drug delivery system having a multi-chamber compartment; and U.S. Pat. No. 4,475,196 which discloses osmotic drug delivery systems. These patent documents are incorporated herein by reference. Many other implants, delivery systems and modules of this type are known to the skilled persons.

In certain cases, the armed NK cells according to the invention can be formulated to ensure good in vivo distribution. For example, the blood-brain barrier (BBB) excludes many highly hydrophilic compounds. To ensure that the therapeutic compounds according to the invention go through the BBB (if so desired), they can be formulated, for example, in liposomes. (See, for example, U.S. Pat. Nos. 4,522,811, 5,374,548 and 5,399,331 for the methods for preparing liposomes). The liposomes can contain one or several substances which are selectively transported to specific cells or organs, and therefore can improve the targeted administration of medicaments (see, for example, VV Ranade (1989) J. Clin. Pharmacol. 29: 685).

### Therapeutic Methods

As previously indicated, said combination of an NK cell allogeneic or autologous with respect to an individual in need thereof, or an NK cell precursor capable of binding to a recombinant polypeptide according to the invention, and said recombinant polypeptide are active ingredients that can be used as a medicament.

According to one embodiment, said NK cell allogeneic or autologous with respect to an individual in need thereof, or an NK cell precursor, and said recombinant polypeptide can be co-administered.

According to one embodiment, said NK cell allogeneic or autologous with respect to an individual in need thereof, or an NK cell precursor, and said recombinant polypeptide can be sequentially administered.

According to one embodiment, said NK cell allogeneic or autologous with respect to an individual in need thereof, or an NK cell precursor, and said recombinant polypeptide can be administered within a non-covalent complex. Therefore, according to one main embodiment, the invention relates to a pharmaceutical composition comprising an NK (Natural Killer) cell or an NK cell precursor, in combination with a recombinant polypeptide; wherein said recombinant polypeptide comprises (i) a modified FEc (fragment crystallizable) region, and (ii) a ligand binding domain, said Fc region being capable of binding to said NK cell or precursor thereof; for use as a medicament.

According to a particular embodiment, the invention relates to a pharmaceutical composition comprising an NK (Natural Killer) cell or an NK cell precursor, in combination with a recombinant polypeptide; wherein said recombinant polypeptide comprises (i) a modified Fc (fragment crystallizable) region, and (ii) a ligand binding domain, said Fc region being bound to said NK cell or precursor thereof; for use as a medicament.

According to a particular embodiment, the invention relates to a pharmaceutical composition comprising an NK (Natural Killer) cell or an NK cell precursor, in combination with a recombinant polypeptide; wherein said recombinant polypeptide comprises (i) a modified Fc region comprising a modified C_{H}2 domain, and (ii) a ligand binding domain, said Fc region being capable of binding to said NK cell or precursor thereof; for use as a medicament.

According to a particular embodiment, the invention relates to a pharmaceutical composition comprising an NK (Natural Killer) cell or an NK cell precursor, in combination with a recombinant polypeptide; wherein said recombinant polypeptide comprises (i) a modified Fc region comprising a modified C_{H}2 domain, and (ii) a ligand binding domain, said Fc region being bound to said NK cell or precursor thereof; for use as a medicament.

According to another main embodiment, the invention relates to an NK cell allogeneic or autologous with respect to an individual in need thereof, or an NK cell precursor attached to a recombinant polypeptide, wherein said recombinant polypeptide comprises (i) a modified Fc (fragment crystallizable) region, capable of binding to said NK cell or precursor thereof, and (ii) a ligand binding domain; for use as a medicament.

According to a particular embodiment, the invention relates to an NK cell allogeneic or autologous with respect to an individual in need thereof, or an NK cell precursor attached to a recombinant polypeptide, wherein said recombinant polypeptide comprises (i) a modified Fc (fragment crystallizable) region, bound to said NK cell or precursor thereof, and (ii) a ligand binding domain; for use as a medicament.

According to a particular embodiment, the invention relates to an NK cell allogeneic or autologous with respect to an individual in need thereof, or an NK cell precursor attached to a recombinant polypeptide, wherein said recombinant polypeptide comprises (i) a modified Fc (fragment crystallizable) region comprising a modified C_{H}2 domain, capable of binding to said NK cell or precursor thereof, and (ii) a ligand binding domain; for use as a medicament.

According to a particular embodiment, the invention relates to an NK cell allogeneic or autologous with respect to an individual in need thereof, or an NK cell precursor attached to a recombinant polypeptide, wherein said recombinant polypeptide comprises (i) a modified Fc (fragment crystallizable) region comprising a modified C_{H}2 domain, bound to said NK cell or precursor thereof, and (ii) a ligand binding domain; for use as a medicament.

According to another main embodiment, the invention relates to a kit comprising:
a first part including an NK cell or an NK cell precursor;
a second part including a recombinant polypeptide comprising (i) a modified Fc (fragment crystallizable) region, and (ii) a ligand binding domain; said Fc region being capable of binding to said NK cell or to an NK cell precursor; for use as a medicament.

According to a particular embodiment, the invention relates to a kit comprising:
a first part including an NK cell or an NK cell precursor;
a second part including a recombinant polypeptide comprising (i) a modified Fc (fragment crystallizable) region comprising a modified C_{H}2 domain, and (ii) a ligand binding domain; said Fc region being capable of binding to said NK cell or to an NK cell precursor; for use as a medicament.

According to another object of the present invention, a pharmaceutical composition according to the invention, an allogeneic or autologous NK cell or NK cell precursor according to the invention or a kit according to the invention is specifically used in a therapeutic method for treating or preventing a cancer, an autoimmune disease and derivatives thereof or an infectious disease in an individual in need thereof.

An individual or patient contemplated according to the invention can be a mammal. A mammal intended by the invention can be selected, for example, from domestic animals (such as bovine, ovine, cats, dogs, and horses), specifically cats and dogs, primates, such as human and non-human primates, rabbits, and rodents, such as mice and rats. According to one embodiment, an individual or patient intended by the invention can be a human being.

An NK cell, preferably allogeneic or autologous with respect to an individual in need thereof, or an NK cell precursor in combination with a recombinant polypeptide according to the invention can be particularly appropriate for use in a method for treating or preventing a cancer, specifically for inhibiting the growth of tumor cells in an individual in need thereof.

According to one embodiment, a cancer or tumor cells associated with the cancer can be part of a melanoma, kidney cancer, prostate cancer, breast cancer, colon cancer, ovarian cancer, lung cancer, bone cancer, pancreatic cancer, skin cancer, head or neck cancer, skin or intraocular malign melanoma, uterine cancer, rectal cancer, anal cancer, stomach cancer, testicular cancer, uterine cancer, Fallopian tube carcinoma, endometrial carcinoma, cervical carcinoma, vaginal carcinoma, vulvar carcinoma, Hodgkin's disease, non-Hodgkin's lymphoma, esophageal cancer, small intestine cancer, endocrine system cancer, thyroid cancer, parathyroid cancer, adrenal gland cancer, soft tissue sarcoma, urethral cancer, penile cancer, chronic or acute leukemias including acute myeloid leukemia, chronic myeloid leukemia, acute lymphoblastic leukemia, chronic lymphocytic leukemia, childhood solid tumors, lymphoid lymphoma, bladder cancer, kidney or urethral cancer, renal pelvis carcinoma, central nervous system (CNS) neoplasm, CNS primary lymphoma, tumor angiogenesis, spinal tumor, brainstem glioma, pituitary adenoma, Kaposi's sarcoma, epidermoid cancer, squamous cell cancer, T-lymphocyte lymphoma, environmentally-induced cancers and combinations of said cancers.

An NK cell allogeneic or autologous with respect to an individual in need thereof, or an NK cell precursor attached to a recombinant polypeptide according to the invention can be particularly appropriate for use in a method for treating or preventing an infectious disease, specifically for inhibiting or destroying cells infected with an infectious pathogen.

According to one embodiment, an infectious disease can be (a) a disease selected from flu, herpes, giardiasis, malaria and leishmaniasis; (b) a pathogen infection with a virus selected from human immunodeficiency virus (HIV), hepatitis virus, herpes virus, adenovirus, influenza virus, flavivirus, echovirus, rhinovirus, coxsackievirus, cornovirus, respiratory syncytial virus, mumps virus, rotavirus, measles virus, rubella virus, parvovirus, vaccinia virus, HTLV virus, dengue virus, papillomavirus, molluscum virus, poliomyelitis virus, rabies virus, JC virus and arboviral encephalitis virus; (c) a pathogen infection with a bacterium selected from Chlamydia, rickettsia bacteria, mycobacteria, staphylococcus, streptococcus, pneumococcus, meningococcus and gonococcus, Klebsiella, Proteus, Serratia, Pseudomonas, Legionella, Diphtheria, Salmonella, Bacilli, cholera, tetanus, botulism, carbon disease, plague, leptospirosis and the Lyme disease bacteria; (d) a pathogen infection with a fungus selected from Candida, Cryptococcus neoformans, Aspergillus, Genus Mucorales, Sporothrix schenka, Blastomyces dermatitidis, Paracoccidioides brasiliensis, Coccidioides immitis and Histoplasma capsulatum; or (e) a pathogen infection with a parasite selected from Entamoeba histolytica, Balantidium coli, Naegleria fowleri, Acanthamoeba sp., Giardia lamblia, Cryptosporidium sp., Pneumocystis carina, Plasmodium virax, Babesia microti, Trypanosoma brucei, Trypanosoma cruzi, Leishmania donovani, Toxoplasma gondii and Nippostrongylus brasiliensis.

Specifically, the hepatitis virus is hepatitis A, hepatitis B or hepatitis C; the herpes virus is VZV, HSV-1, HAV-6, HSV-II and CMV or Epstein-Barr virus; the fungus Candida is Candida albicans, Candida krusei, Candida glabrata or Candida tropicalis; the fungus Aspergillus is Aspergillus fumigatus or Aspergillus niger; and the fungus Mucorales is mucor, absidia or rhizophus.

An NK cell, preferably allogeneic or autologous with respect to an individual in need thereof, or an NK cell precursor attached to a recombinant polypeptide according to the invention can also be particularly convenient for use in a method for treating or preventing an autoimmune disease or derivatives thereof, specifically a primitive or secondary autoimmune and/or inflammatory disease, specific to organs or systemic, and associated or not to pathogenic auto-antibodies.

By way of example, the following can be mentioned: organ graft rejection, graft-versus-host disease, rheumatoid arthritis, systemic lupus erythematosus, scleroderma, primitive Sjôgren's syndrome (or Gougerot-Sjôgren's syndrome), autoimmune polyneuropathies such as multiple sclerosis, type-I diabetes, autoimmune hepatitis, ankylosing spondylitis, Reiter's syndrome, gouty arthritis, celiac disease, Crohn's disease, Hashimoto's thyroiditis, Addison's disease, autoimmune hepatitis, Basedow's disease, ulcerative colitis, vasculitis such as ANCA (antineutrophil cytoplasmic antibodies)-associated systemic vasculitis, autoimmune cytopenias and other hematologic complications in adults and children, such as acute or chronic autoimmune thrombopenia, autoimmune hemolytic anemias, hemolytic disease of the newborn (HDN), cold agglutinin disease, thrombotic thrombocytopenic purpura and autoimmune acquired hemophilia, Goodpasture syndrome, extramembranous nephropathy, autoimmune oily skin conditions, refractory myasthenia, mixed cryoglobulinemias, psoriasis, juvenile idiopathic arthritis, inflammatory myositis, dermatomyositis and systemic autoimmune conditions in children including antiphospholipid syndrome.

A method is also provided for treating and/or preventing a cancer, an autoimmune disease and derivatives thereof or an infectious disease in an individual in need thereof, comprising the administration to the individual of an armed NK cell according to the invention in combination with or without an antigen specific for cancer, an autoimmune disease and derivatives thereof or the infectious disease to be cured in the individual.

Other than the therapies according to the invention, the armed NK cells according to the invention can also be used in combination with another therapy. For example, for the treatment of cancer, the armed NK cells according to the invention can be administered to an individual who also receives another treatment against cancer, such as chemotherapy, radiotherapy, surgery or genetic therapy.

### EXAMPLES

The following examples show the binding specificity between eNK cells and a modified Fc polypeptide, particularly an Fc SDH polypeptide (Fc-SDHTSFTIE), and the capacity thereof to target cells involved in diseases.

### Example 1: Material and Methods

### Preparation of PBMC Cells

The PBMC cells (T-cells, B-cells, NK cells and monocytes) were obtained from healthy donors of CHU in Montpellier. The cells were taken from peripheral blood samples (B-cells, T-cells, and monocytes) and from umbilical cord blood (NK cells) (Sanchez-Martinez et al. 2016, Sanchez-Martinez et al. 2018).

### PBMC Cells Markers

CD45⁺ is a general marker for lymphocytes. CD56⁺ is a specific marker for NK cells. CD3⁻ is a marker distinguishing T-lymphocytes. CD16⁺ is a specific marker for NK cells, allowing antibody-dependent cellular cytotoxicity (ADCC).

### Isolation and Activation of NK Cells

First, the lymphocytes were obtained from healthy donors of CHU in Montpellier. PBMC and UCB (UCBMC) were respectively taken in peripheral blood samples and UCB units using Histopaque-1077 (Sigma). In essence, 13 ml of Histopaque were added to 50 ml of centrifugation tubes and 30 ml of blood diluted 1/2 in RPMI (Invitrogen) were slowly added from the top. The tubes were centrifuged at 1600 revolutions/minute for 30 minutes at 20° C. without interruption.

After centrifugation, the mononuclear cells were taken from the white interlayer ring, washed in RPMI and in suspension in the RPMI medium supplemented with 10% of FBS (Invitrogen). The lymphocytes were then frozen with liquid nitrogen. The frozen lymphocytes were then depleted of T-cells using the positive selection kit CD3 EasySep™ (STEMCELL technologies). The cells were cultured for 10 to 20 days with γ-irradiated PLH cells at an NK cell/irradiated PLH cell ratio of 1:1 in the presence of IL-2 (100 U/ml) and IL-15 (5 ng/ml), or with IL-2 only (1000 U/ml). PLH cells were added every four days and fresh cytokines every two days. At the end of the treatment, the purity of the NK cells (CD56⁺/CD3⁻) was always greater than 90%.

### IgG1 Antibodies Comprising a Modified Fc Region

The IgG1 antibody used is trastuzumab (Herceptin) or rituximab (Rituxan). The modified Fc regions are listed in the following table.

TABLE 1

| | Designation | | EU 231-340 |
|---|---|---|---|
| Designation #1 | #2 | IgG | sequence |
| Fc WT (wild type) | WT | IgG1-C_{H}2 | SEQ ID NO. 1 |
| Fc SDH | L | IgG1-C_{H}2 | SEQ ID NO. 2 |
| Fc SD | A | IgG1-C_{H}2 | SEQ ID NO. 3 |
| Fc IE | B | IgG1-C_{H}2 | SEQ ID NO. 4 |
| Fc SDIE | C | IgG1-C_{H}2 | SEQ ID NO. 5 |
| Fc SDSTIE | K | IgG1-C_{H}2 | SEQ ID NO. 6 |
| Fc SDHFIE | J | IgG1-C_{H}2 | SEQ ID NO. 7 |
| Fc AL | D | IgG1-C_{H}2 | SEQ ID NO. 8 |
| Fc ALIE | E | IgG1-C_{H}2 | SEQ ID NO. 9 |
| Fc GASD | F | IgG1-C_{H}2 | SEQ ID NO. 10 |
| Fc GASDALIE | G | IgG1-C_{H}2 | SEQ ID NO. 11 |
| Fc GASDIE | H | IgG1-C_{H}2 | SEQ ID NO. 12 |
| Fc SDALIE | I | IgG1-C_{H}2 | SEQ ID NO. 13 |
| Fc LALA | M | IgG1-C_{H}2 | SEQ ID NO. 14 |
| Fc WT (whole) with | | IgG1-Fc | SEQ ID NO. 15 |
| nG1ml allotype | | | |
| Fc WT (whole) with | | IgG1-Fc | SEQ ID NO. 16 |
| G1m17,1 allotype | | | |
| Fc WT (whole treated | | IgG1-Fc | SEQ ID NO. 17 |
| with papaine) with | | | |
| G1m17,1 allotype | | | |

The amino acid sequence SEQ ID NO. 17 is encompassed in the amino acid sequence SEQ ID NO. 16. The difference of said two amino acid sequences resides in that the first 10 amino acids of the amino acid sequence SEQ ID NO. 16 are removed from the amino acid sequence SEQ IS NO. 17.

Labeling of the Fc polypeptides and of the Fc antibodies with fluorescent molecules In order to label the Fc polypeptides and the Fc antibodies and study their binding to NK cells, the polypeptides and antibodies are linked to a fluorescence molecule. The fluorescent molecules used are AlexaFluors 647 (A647) and AlexaFluors 488 (A488), fluorescein isothiocyanate (FITC).

### Flow Cytometry Analysis

For the analysis of the phenotype, the cells were dyed with 7AAD (Beckman) in order to identify viable cells and antibodies against surface markers CD25-FITC, CD45RO-FITC, CD69-PE, CD62L-PE, CD19-PE, CD3-PE, CD19-ECD, CD56-PECy7, CD56-APC, CD3-APC, CD45-APCAlexaFluor750, CD45RA-APCAlexaFluor750, CD16-PacificBlue, CD57-PacificBlue, CD45-KromeOrange, CD16-KromeOrange (Beckman), CD158b-FITC, CD158a-PE, CD107a-HV500 (BD Biosciences), CD158e-Vioblue (Miltenyi). 1×10⁵-3×10⁵ cells were incubated for 20-30 minutes at 4° C. with different antibodies in PBS containing 2.5% of PBS. The cells were then washed and placed in suspension in 200-250 μl of the same medium. The dyeing was analyzed by Gallios flow cytometry (Beckman) by means of Kaluza software. Live lymphocytes were subjected to FSC/SSC and 7AAD dyeing. B-lymphocytes (CD19⁺), T-lymphocytes (CD3⁺/CD56⁻) and NK cells (CD56⁺/CD3⁻) were distinguished using the CD19, CD3 and CD56 antibodies, respectively.

### MTT Test

The NK cells freshly taken or frozen (preserved in liquid nitrogen) were labeled with 3 μM of CellTracker™ Violet BMQC Dye (Life Technologies) and incubated overnight with BT20 target cells at different E:T ratios. Thereafter, the translocation of phosphatidylserine (PS) and membrane damage were analyzed in the population of target cells negative for violet fluorescence by flow cytometry using Annexin V-FITC (Immunostep) and 7AAD (BD Biosciences) or propidium iodide (PI). All cells positive for Annexin-V and/or PI (or 7-AAD) were considered as dead (or dying).

In the ADCC experiments, BT20 target cells were incubated with the antibody trastuzumab at 5 g/ml for 30 minutes at 37° C. When the NK cells are armed with the antibodies, the cells are incubated at the same antibody concentration before washing them and incubating them with BT20 target cells. EGTA (ethylene glycol-bis(2-aminoethylether)-N,N,N′,N′-tetraacetic acid) was used at 1 mM to block the granular exocytose route and MgCl₂ at 1.5 mM to maintain the osmotic pressure. Tetrazolium MTT (3-(4,5-dimethylthiazol-2-yl)-2,5-diphenyltetrazomium bromide) dye was used to determine cell viability. Then, 10 μL of MTT (5 mg/ml) were added to adherent cells (100 μL of medium after 2 washings with PBS) and the cells were then incubated for 1 h at 37° C. After 1 h, 100 L of 0.05 M HCl were added to isopropanol to dissolve the crystals and quantify the color at 550 nm in a spectrophotometer. In all the experiments, basal cell death was calculated in the absence and in the presence of monoclonal antibodies. These values were subtracted from those obtained after the NK cell or treatment with NK cells armed with antibodies to generate specific ADCC, respectively.

### Statistical Analysis

All the experiments were performed with BD FACSCANTO II and analyzed with Flowjo software.

### Example 2 Study Design

### Combination of Fc Polypeptide and eNK Cells (Graph 1A)

in vitro NK cells from cord blood (eNK) were incubated with the polypeptides (i) Fc LALA A647, (ii) Fc WT A647, (iii) Fc SDH A647 at 10 μg/ml or without Fc for 1 h at 37° C. The eNK cells were then washed and dyed with respect to NK markers. Armed eNK cells were then identified by the surface markers CD45⁺/CD56⁺/CD3⁻ by means of flow cytometry. The mean fluorescent intensity (MFI) of the different Fc polypeptides labeled with A647 (Fc A647) was analyzed and normalized to Fc WT polypeptide.

### Combination of Fc Trastuzumab Antibody and eNK Cells (Graph 1B)

eNK cells were incubated with Fc WT trastuzumab antibody, Fc SDH trastuzumab antibody at 10 g/ml, or without Fc antibody for 1 h at 37° C. The eNK cells were then washed and labeled with a secondary anti-human Fc IgG antibody coupled to FITC (anti-Fc IgG FITC). The armed eNK cells were then identified by the surface markers CD45⁺/CD56⁺/CD3⁻ by means of flow cytometry. The MFI of the different anti-Fc IgG FITC was analyzed and normalized to Fc WT.

### Binding Specificity of Fc Polypeptide and Fc Receptor (CD16) of the eNK Cells (Graph 1C)

eNK cells were incubated with the polypeptides (i) Fc LALA A647, (ii) Fc WT A647, (iii) Fc SDH A647 at 10 μg/ml or without Fc for 1 h at 37° C. The eNK cells were then washed and dyed with respect to the NK markers. The armed eNK cells were then identified by the surface markers CD45⁺/CD56⁺/CD3⁻ and CD16⁺ or CD16⁻ by means of flow cytometry.

The frequency of Fc⁺ eNK cells and the MFI (normalized to Fc WT) were analyzed in the CD16⁻ NK and CD16⁺ NK cells.

### Example 3 Study Design

eNK cells were incubated with the polypeptides (i) Fc LALA A647, (ii) Fc WT A647, (iii) Fc SDH A647 at 10 μg/ml or without Fc for 1 h at 37° C. The eNK cells were then washed and incubated at 37° C. for 2, 3 or 7 days. On the indicated days, the binding of the Fc polypeptide on the CD16⁺/CD56⁺/CD45⁺/CD3⁻ eNK cells was analyzed by flow cytometry (graph 2A). The frequency of Fc⁺ eNK cells and the MFI (normalized to Fc WT) of Fc A647 (graph 2B) were measured on the eNK cells.

### Example 4 Study Design

### Dose of Fc SDH for an Efficient Attachment (Graph 3A and Graph 3B)

In order to test if all the CD16A receptors (or FcγRIIIa) can be saturated by the Fc SDH polypeptide, eNK cells were incubated with increasing concentrations of Fc WT polypeptide or Fc SDH polypeptide.

Specifically, eNK cells were incubated with the polypeptides (i) Fc WT A647, (ii) Fc SDH A647 or (iii) Fc SDH A488 at 1, 10, 20, 30, 40 μg/ml, or without Fc for 1 h at 37° C. The eNK cells, once armed with the Fc polypeptides, were washed and dyed with respect to the NK markers. The armed eNK cells were identified as CD45⁺/CD56⁺/CD3⁻ by flow cytometry. The MFI of Fc A647 (graph 3A) or Fc A488 (graph 3B) was analyzed.

### Combination of the Fc SDH Polypeptide and Competitors (Graph 3C and Graph 3D)

The examples described above show the long-term stability of the Fc SDH polypeptide on eNK cells, but whether or not this attachment can be displaced by another Fc polypeptide when injected in vivo remains to be answered. In order to study this condition, eNK cells were incubated under saturating conditions with polypeptides Fc SDH A488 (graph 3C) or Fc SDH A647 (graph 3D) at 20 μg/ml, or without Fc for 1 h at 37° C. The eNK cells, once armed with Fc polypeptides, were then washed and ligands of the CD16A receptor competitors were added for 1 h at 37° C.: (i) Fc block; (ii) Fc WT; (iii) Fc SDH; anti-CD16 antibody. The eNK cells were then dyed with respect to the NK markers. The eNK cells were defined as CD45⁺/CD56⁺/CD3⁻ by flow cytometry. The MFI of Fc A488 (graph 3C) or Fc A647 (graph 3D) was analyzed.

### Example 5 Study Design

Cancer Cell Survival in the Presence of eNK Cells Armed with Fc SDH Antibodies (Graphs 4A, 4B, and 4C)

The eNK cells were incubated with Fc WT trastuzumab antibody or Fc SDH trastuzumab antibody at g/ml or 1 μg/ml, or without antibody for 1 h at 37° C. The eNK cells armed with Fc antibodies were then washed (graphs 4B and 4C) or not washed (graph 4A), incubated for 1 h (graphs 4A and 4B) or for 24 h (graph 4C) at 37° C., then placed in the presence of BT20 cells at a 1:1 ratio for 24 h at 37° C. Cell viability, represented by the survival percentage, was measured by means of an MTT test.

### Non-Specific Activation by Armed eNK Cells (Graph 4D)

The eNK cells were incubated with WT trastuzumab antibody or SDH trastuzumab antibody at 10 g/ml or 1 μg/ml, or with no antibody for 1 h at 37° C. The eNK cells armed with antibodies were then washed, incubated for 1 h at 37° C., then placed in the presence of BT20 cells at a 1:1 ratio with antibodies for detecting CD107a at 37° C. After 1 h of incubation, monensin and brefeldin A-type compounds were added for an additional 5 h in order to block the degradation of CD107a. The analysis of the expression of CD107a on the CD56⁺/CD45⁺/CD3⁻ eNK cells was performed by flow cytometry.

### Example 6 Study Design

eNK cells were incubated with Fc WT or Fc SDH antibodies at 10 μg/ml or without antibody for 1 h at 37° C. The eNK cells were then washed and split into 2 samples. The armed eNK cells from the first sample were labeled with an anti-Fc IgG for the detection of antibodies present at the membrane surface. The eNK cells from the second sample were fixed and permeabilized before being labeled with an anti-Fc IgG in order to detect the antibodies present at the membrane surface and in the intracellular medium. The armed eNK cells were then identified by the surface markers CD45⁺/CD56⁺/CD3⁻ by means of flow cytometry. The mean fluorescent intensity (MFI) of the different Fc antibodies labeled with the anti-Fc IgG was measured (graph 5A).

The eNK cells were incubated with polypeptides Fc SDH A488 (graph 5B) or Fc SDH A647 (graph 5C) at 20 μg/ml, or without Fc for 1 h at 37° C. The eNK cells were then washed and incubated at 37° C. for 3 days. Thereafter, the competitor CD16A ligands were added for 1 h at 37° C.: (i) Fc block; (ii) anti-CD32 antibody; (iii) anti-CD16 clone B73.1, (iv) anti-CD16 antibody. The cells were dyed with respect to the NK markers. The eNK cells were defined as CD45⁺/CD56⁺/CD3⁻ by flow cytometry and the MFI of Fc A488 (graph 5B) or Fc A647 (graph 5C) was analyzed.

### Example 7 Study Design

Previously frozen eNK cells were incubated with Fc SDH A647 polypeptides a 10 μg/ml for 1 h at 37° C. The eNK cells were then washed and 15·10⁶ eNK were injected intraperitoneally into an adult Swiss-nude mouse. The mouse was sacrificed 24 hours later and peritoneal fluid and several organs (blood, bone marrow, and spleen) were removed for analysis (FIGS. 7A, 7B, 7C, and 7D). The cells were dyed to detect the NK markers. Using flow cytometry, the eNK cells were defined as CD45⁺/CD56⁺/CD16^{low} or CD16⁺ and the Fc A647 fluorescence was analyzed in peritoneal cavity cells. Comparison of Fc A647⁺ eNK cells within total CD56⁺ eNK cells before and after in vivo injection (FIG. 7B).

### Example 8 Study Design

8.1. In Vitro Binding Affinity of Tested Recombinant Antibodies Having a Modified C_{H}2 on their Fc Region Compared to a Wild-Type Antibody to CD16a and CD16b Receptors on NK Cells (FIG. 9A).

The binding properties of the tested recombinant antibodies on CD16a or CD16b were compared to a wild-type antibody without amino acid mutations on its Fc region, in particular its C_{H}2 sequence (using the rituximab monoclonal antibody, allotype G1m17,1) by SPR (surface plasmon resonance) (FIG. 9A).

Anti-polyhistidine antibodies (R&D Systems) were immobilized on a T200 apparatus at 25° C. in HBS-EP at 10 l/min flow rate on a CM5 sensor chip using EDC/NHS activation, according to the manufacturer's instructions (GE Healthcare). They were covalently immobilized at the 6700RU level on the flowcell Fc2 and a control reference surface (flowcell Fc1) was prepared using the same chemical treatment but without antibody. All kinetic measurements in Fc1 and Fc2 were performed by single-cycle titration on a T200 apparatus at 25° C. in HBS-EP at 100 μl/min. Each human gamma receptor (R&D Systems) was captured on immobilized anti-polyhistidine antibody at 20 nM during 60 s. Five increasing concentrations of antibody were injected (injection time=120 s). After a dissociation step of 600 s in running buffer, sensor surfaces were regenerated using 5 μl of glycine-HCl pH1.7. All the sensorgrams were corrected by subtracting the low signal from the control reference surface and buffer blank injections. Kinetic parameters were evaluated from the sensorgrams using a two states models from the T200 evaluation software.

The results are normalized regarding the affinity of the WT antibody on the receptor.

8.2. Kinetics of Binding on CD16+ Cells of the Tested Recombinant Antibodies from 1 h to 72 h after CD16 Arming (FIG. 9B).

The non-covalent binding kinetics at the cell surface of CD16+ cells (i.e., NK cells) was evaluated by FACS. eNK were incubated with rituximab WT or carrying recombinant antibodies at 10 μg/mL for 1 hour at 37° C. NK cells were then washed and incubated at 37° C. for 1 h, 24 h, 48 h or 72 h.

Flow cytometry was used to analyze mAbs binding, using an anti-idiotype antibody, on CD56+/CD45+/CD16+/CD3− eNK cells. Mean of % armed NK cells is reported on the graph. All experiments were performed with a BD FACSCANTO II and analyzed with Flowjo software.

### Example 9 Study Design

eNK cells were incubated with recombinant rituximab SDH (RTX-Fc SDH) and/or recombinant trastuzumab SDH (TRAST-Fc SDH) at 10 μg/mL for 1 hour at 37° C.

NK cells were then washed and analyzed by flow cytometry for Ab binding, using an anti-idiotype antibody, on CD56+/CD45+/CD16+/CD3− eNK cells. All experiments were performed with a BD FACSCANTO II and analyzed with Flowjo software.

### Example 10 Study Design

CD16+NK cells were armed with recombinant RTX-SDH or not (Fc WT), then incubated with B-lymphoma primary cells from mantle lymphoma patients (1, 2, 3), DLBCL patients (4, 5), follicular lymphoma patients (6, 7) at ratio E:T 3:1 for 16 h.

Flow cytometry was used to determine the percentage of ADCC (antibody-dependent cellular cytotoxicity) of armed CD16+NK cells after subtraction of NK cells natural cytotoxicity (n=2 to 4 eNK donors tested, dots). Bars represent mean±SEM. Whatever the B-lymphoma type and the patient, CD16+ cells armed with RTX-SDH kill more target cells than the CD16+ cells alone (from 20% to 70% more cytotoxic effect).

### Results

### Example 2: The Attachment of Fc SDH Polypeptide is More Efficient than that of Wild Type Fc (WT)

A weak attachment of the Fc LALA polypeptide to eNK cells (graph 1A) and of the Fc WT antibody to eNK cells (graph 1B) can be observed. On the other hand, a strong attachment of the Fc SDH polypeptide to eNK cells is observed compared with the positive control (Fc WT) whether with respect to the isolated polypeptide (graph 1A) or to the Fc region of an IgG antibody (graph 1B).

Furthermore, it has been noted that the fluorescent intensity of the Fc SDH polypeptide is significantly higher in the CD16⁺ eNK cell population (30) with respect to the CD16⁻ eNK cell population (3) (graph 1C, left). Also, the percentage of Fc⁺ eNK cells is significantly higher in the CD16⁺ eNK cell population armed with Fc SDH (100%) with respect to the CD16⁻ eNK cell population armed with Fc SDH (40%). Therefore, the attachment of the Fc SDH region of a polypeptide is highly specific for the CD16⁺ eNK cell population (graph 1C), which suggests that the main Fc receptor used by Fc WT or Fc SDH is CD16A.

The binding specificity of Fc SDH polypeptide-eNK cells is greatly improved with respect to the attachment of the Fc WT polypeptide as a result of SDH mutations (40% of CD16⁺ eNK cells armed with Fc WT versus 100% of the CD16⁺ eNK cells armed with Fc SDH, graph 1C, right).

The eNK cells armed with Fc SDH attached to fluorescent molecules illustrate the capacity of these armed eNK cells to bind to any small molecule, for example antigens of interest, allowing the recognition and removal of target cells involved in diseases.

### Example 3: The Attachment of the Fc SDH Polypeptide is Stable Over Time

The flow cytometry analysis shows that the eNK cells are practically depleted of Fc LALA and Fc WT polypeptides after 2 days of incubation compared to the negative control (no Fc). On the contrary, the Fc SDH polypeptide remains strongly attached to the eNK cells after 7 days of incubation (graph 2A).

Furthermore, it is noted that more than 80% of the eNK cells still carry the Fc SDH polypeptide after 7 days whereas the attachment of the Fc WT polypeptide is very weak by the 2^{nd} day of incubation with about 10% of the cells still carrying the Fc WT polypeptide (graph 2B).

In conclusion, the attachment between Fc SDH-eNK cells remains extremely stable at least until 7 days of incubation.

### Example 4: The Combination of Fc SDH-NK Cells is Saturating and Cannot be Displaced Under Normal Physiological Conditions

A low dose of Fc SDH polypeptide is required to saturate the NK cell receptors.

The Fc SDH A647 polypeptide quickly saturates the CD16A sites starting at 10 μg/ml whereas the Fc WT A647 polypeptide does not achieve saturation even at 40 μg/ml (graph 3A). Similarly, the Fc SDH polypeptide attached to a fluorescent molecule other than A488 saturates the eNK cells starting at 10 μg/ml (graph 3B).

Therefore, a low dose of Fc SDH polypeptide is required to saturate the NK cells.

The attachment of the Fc SDH polypeptide cannot be removed under physiological conditions.

The data shows that two forms of wild type Fc polypeptide (the Fc block and the Fc WT A647) have not modified the level of attached Fc SDH polypeptide. On the contrary, another Fc SDH polypeptide lowered the level of Fc SDH initially attached to the two combinations of fluorochromes A488 and A647, due to the same affinity for CD16A. The largest displacement was observed when the anti-CD16 antibody was used, which implies that the attachment of Fc SDH depends on the CD16A receptor (graphs 3C and 3D).

Therefore, once attached to CD16A, the Fc SDH polypeptide cannot be removed by the Fc WT polypeptides, which suggests that, when the eNK cell armed with the Fc SDH polypeptide is injected in vivo, the Fc SDH polypeptide remains stable and attached to the NK cell.

### Example 5: eNK Cells Armed with Fc SDH Antibodies are More Cytotoxic and Show High Degranulation Levels

eNK cells armed with Fc SDH antibodies exhibit greater cytotoxicity than an eNK cell alone or armed with Fc WT antibodies

In order to test the cytotoxicity of armed eNK cells, it was chosen to use a modified trastuzumab antibody specific for the HER2 protein expressed at the surface of cancer cells, including the BT20 breast cancer cell line. These cell lines will be the target for the armed eNK. The results show that the eNK cells armed with an Fc SDH antibody at 10 μg/ml are more cytotoxic than eNK cells alone or armed with Fc WT antibodies at 10 μg/ml (graphs 4A and AB). The same results are obtained with eNK cells armed for 24 h with Fc SDH antibodies (graph 4C). The dosage of the antibodies at 1 g/ml has shown a less potent effect. The Fc WT antibody does not seem to have any effect with respect to the eNK cell alone (no antibody). Therefore, the cytotoxicity level of eNK cells is improved with Fc SDH antibodies.

High Degranulation Level for the eNK Cells Armed with Antibodies Fc SDH

The cytotoxicity of eNK cells is mediated by degranulation. This mechanism can be measured by the expression of CD107a. Membrane expression of CD107a is a marker for the cytotoxic activity of NK cells. Therefore, the expression of CD107a was analyzed by flow cytometry on eNK cells armed with Fc WT antibody or with Fc SDH antibody in the presence or absence of target cells (BT20 cells). The results show that CD107a is not induced on the armed eNK cells without target cells (graph 4C, top), which suggests that the attachment of antibodies to CD16A does not entail the activation of armed eNK cells in the absence of target cells. Second, the eNK cells armed with Fc SDH antibody at 10 μg/ml show a higher CD107a expression than that of Fc WT antibody or in the absence of antibodies (graph 4D, bottom).

One of the mechanisms used by eNK cells armed with Fc antibody is degranulation and this mechanism is more effective on eNK cells armed with Fc SDH antibody.

### Example 6: The Fc SDH Antibody Attached to the Fc Receptor (CD16) of eNK Cells is not Internalized

It was noted that, after 3 days of incubation, the Fc SDH A647 antibody or the Fc SDH A488 antibody is removed by a competing anti-CD16 antibody. On the other hand, the anti-CD32 antibody does not displace the Fc SDH antibody initially attached to the Fc receptor. Therefore, it has been noted that the Fc SDH antibody remains present on the surface of the eNK cells and thus it is not internalized (FIG. 5).

In addition, several studies have reported an excretion or internalization of CD16 receptors during activation of NK cells (Romee et al., 2013; Capuano et al., 2017). In order to measure if the attachment to the Fc region disrupts the expression of CD16A receptor, the anti-CD16 clone B73.1 was used, the attachment of which is not supposed to be affected by the masking of Fc since the epitope thereof is in the first Ig-type distal membrane domain of the CD16 molecule (Grier et al., 2012). However, it can be seen in graphs 3C and 3D that this antibody can displace the Fc SDH antibody. Therefore, the results of FIG. 5 show that the frequency of CD16⁺ eNK cells does not vary over time and according to the conditions of Fc.

### Example 7: The Attachment of the Fc SDH Region on the eNK Cells is Stable In Vivo

In order to ensure the binding stability of Fc SDH-eNK cells under in vivo conditions, eNK cells armed with Fc SDH polypeptides were injected intraperitoneally into an adult Swiss-nude mouse. It has been noted that many eNK cells (15% of all cells) are found in the peritoneal cavity (FIG. 7A). These cells were still largely CD16⁺ (79%), and most of the CD16⁺ eNK cells were armed with Fc SDH A647 polypeptides (87%). Before the injection, 84% of the total eNK cells were provided with Fc SDH A647 polypeptides, and 73% of them remained positive after 24 h in vivo (FIG. 7B). Furthermore, 24 h after the injection, it was noted that a small number of eNK cells are present in the blood or the bone marrow (FIG. 7C). However, some CD16⁺ eNK cells have migrated towards the spleen and remain armed with Fc SDH A647 (88%) in the spleen (FIG. 7D).

Therefore, these results show that the attachment of Fc SDH remains stable on the eNK cells after injection in vivo.

### Example 8

### 8.1. In Vitro Binding Affinity of Different C_{H}2 Mutants Compared to the Wild-Type Form to CD16A and CD16B (FIG. 9A).

FIG. 9A represents the affinity of the tested recombinant antibodies against CD16a (light grey) and CD16b (dark grey) compared to the reference value antibody RTX-Fc WT. The data demonstrates that except for recombinant antibody D (Fc AL—SEQ ID NO. 8), all the other recombinant antibodies show improved in vitro binding to the CD16a and CD16b receptors (FIG. 9A).

Among all, the recombinant antibodies tested, C (Fc SDIE—SEQ ID NO. 5), I (Fc SDALIE—SEQ ID NO. 13), J (Fc SDHFIE—SEQ ID NO. 7), K (Fc SDSTIE—SEQ ID NO. 6) and L (Fc SDH—SEQ ID NO. 2) show the highest binding to CD16a with close or more than 8-fold increase in affinity compared to the WT antibody. Recombinant antibodies E (Fc ALIE—SEQ ID NO. 9), G (Fc GASDALIE—SEQ ID NO. 11) and H (Fc GASDIE—SEQ ID NO. 12) show 5-6-fold increased binding and recombinant antibodies A (Fc SD—SEQ ID NO. 3), B (Fc IE—SEQ ID NO. 4), and F demonstrate a smaller improvement with 2-3-fold increase binding to CD16a.

Specifically, recombinant antibodies J, K and L share the same mutations S239D and I332E and one or two additional mutations show a remarkable binding affinity compared to all the other recombinant antibodies including other recombinant antibodies C, G, H or I sharing the same mutations S239D and I332E. These additional mutations in recombinant antibodies J, K and L may thus confer an advantage when bringing these antibodies into contact with CD16+ cells and/or NK cells.

Recombinant antibody L (Fc SDH—SEQ ID NO. 2) includes mutations S239D, H268F, S324T and I332E. Mutation H268F is included by recombinant antibody J whereas mutation S324T is included by recombinant antibody K. Therefore, mutations H268F and S324T also allow to increase the binding affinity of the antibody on the CD16a receptor.

The results on CD16b are relatively similar except that it is observed a CD16b improved binding with mutants C and L at a lesser extent.

8.2. Kinetics of Binding on CD16+ Cells of the Tested Recombinant Antibodies Having Modified C_{H}2 on Fc Region from 1 h to 72 h after CD16 Arming (FIG. 9B).

FIG. 9B represents the percentage of CD56⁺/CD45⁺/CD16⁺/CD3⁻ eNK cells coated with RTX-Fc WT or each recombinant antibody (RTX) on their surface, at 1 h, 24 h, 48 h or 72 h after arming. Less than 20% of CD16+ cells were coated after 1 hour with the RTX-FcWT and the recombinant antibody D, and from 24 hours most of the NK cells (CD16+ cells) do not present the antibody on their surface (FIG. 9B).

All the other recombinant antibodies are able to bind on more than 50% CD16+ cell (i.e., NK cells) at 1 hour but discrepancies are observed regarding their kinetics of binding. The binding of recombinant antibodies A, B, E and F drastically decreases at 24 hours with more than 50% loss compared to 1 hour. This is not the case for recombinant antibodies C, G, H, I, J, K and L for which a stable binding is observed overtime and until 72 hours with more than 50% CD16+ cells remaining coated with the antibodies compared to 1 hour. One would anticipate that recombinant antibodies with the best affinity for CD16 would be the best armed on CD16+ cells and inversely. But, while presenting a higher affinity for the CD16 than the mutant H and the mutant G, the mutant E presents a less stable binding on CD16+ cells.

It must be noted that recombinant antibodies I, J and K, that share the same mutations S239D and I332E with one additional mutation, have a durable and stable persistence binding to CD16 receptor between the recombinant antibody and the NK cell (CD16+ cell).

It is also important to note that the allotype has no impact on the long-term stable binding of the recombinant antibodies as demonstrated by comparing the RTX-L-R (G1m17,1 allotype) and the RTX-L-T (nG1m1 allotype) on FIG. 9B.

This data demonstrates that the unexpected long-term binding (more than 72 hours, FIG. 9B) of different recombinant antibodies on CD16+ cells cannot be anticipated by their receptor affinity improvement (FIG. 9A).

### Example 9: Multi-Arming Efficiency on CD16+ Cells

The results depicted in FIG. 10 show that CD16+ cells (i.e., NK cells) can be co-armed with two mAb each mutated on their CH2 domain. This double arming has the same binding efficiency as single arming.

### Example 10: Increase Cytotoxicity of CD16+ Cells Armed with Recombinant Rituximab Fc SDH

The results depicted in FIG. 11 demonstrate that the ex vivo armament of CD16+ cells (i.e., NK cells) with a monoclonal antibody containing a Fc SDH mutation (represented in SEQ ID NO. 2) can lead to strong ADCC on targeted cancer cells.

### LIST OF CITED DOCUMENTS

Capuano, C., C. Pighi, R. Molfetta, R. Paolini, S. Battella, G. Palmieri, G. Giannini, F. Belardinilli, A. Santoni & R. Galandrini (2017) Obinutuzumab-mediated high-affinity ligation of FcγRIIIA/CD16 primes NK cells for IFNγ production. Oncoimmunology, 6, e1290037.
Grier, J. T., L. R. Forbes, L. Monaco-Shawver, J. Oshinsky, T. P. Atkinson, C. Moody, R. Pandey, K. S. Campbell & J. S. Orange (2012) Human immunodeficiency-causing mutation defines CD16 in spontaneous NK cell cytotoxicity. J Clin Invest, 122, 3769-80.
Moore, G. L., H. Chen, S. Karki & G. A. Lazar (2010) Engineered Fc variant antibodies with enhanced ability to recruit complement and mediate effector functions. MAbs, 2, 181-9.
Romee, R., B. Foley, T. Lenvik, Y. Wang, B. Zhang, D. Ankarlo, X. Luo, S. Cooley, M. Verneris, B. Walcheck & J. Miller (2013) NK cell CD16 surface expression and function is regulated by a disintegrin and metalloprotease-17 (ADAM17). Blood, 121, 3599-608.
Sanchez-Martinez, D., N. Allende-Vega, S. Orecchioni, G. Talarico, A. Cornillon, D. N. Vo, C. Rene, Z. Y. Lu, E. Krzywinska, A. Anel, E. M. Galvez, J. Pardo, B. Robert, P. Martineau, Y. Hicheri, F. Bertolini, G. Cartron & M. Villalba (2018) Expansion of allogeneic NK cells with efficient antibody-dependent cell cytotoxicity against multiple tumors. Theranostics, 8, 3856-3869.

### Sequence Listing

SEQ ID NO. 1 (= Fc WT region (IgG1-C_{H}2))
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
SEQ ID NO. 2 (= Fc SDH region (IgG1-C_{H}2.S239D.H268F.S324T.I332E))
APELLGGPDVFLFPPKPKDTLMISRTPEVTCVVVDVSFEDPEVKFNWYVDGVEVHNAKTKP
REEQYNSTYRVVSVLTVLHQDWLNGKEYKCKVTNKALPAPEEKTISKAK
SEQ ID NO. 3 (= Fc SDH region (IgG1-C_{H}2.S239D))
APELLGGPDVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
SEQ ID NO. 4 (= Fc IE region (IgG1-C_{H}2.I332E))
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPEEKTISKAK
SEQ ID NO. 5 (= Fc SDIE region (IgG1-C_{H}2.S239D.I332E))
APELLGGPDVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPEEKTISKAK
SEQ ID NO. 6 (= Fc SDSTIE region (IgG1-C_{H}2.S239D.S324T.I332E))
APELLGGPDVFLFPPKPKDTLMISRTPEVTCVVVDVSHEDPEVKFNWYVDGVEVHNAKTKP
REEQYNSTYRVVSVLTVLHQDWLNGKEYKCKVTNKALPAPEEKTISKAK
SEQ ID NO. 7 (= Fc SDHFIE region (IgG1-C_{H}2.S239D.H268F.I332E))
APELLGGPDVFLFPPKPKDTLMISRTPEVTCVVVDVSFEDPEVKFNWYVDGVEVHNAKTKP
REEQYNSTYRVVSVLTVLHQDWLNGKEYKCKVSNKALPAPEEKTISKAK
SEQ ID NO. 8 (= Fc AL region (IgG1-C_{H}2.A330L))
APELLGGPDVFLFPPKPKDTLMISRTPEVTCVVVDVSHEDPEVKFNWYVDGVEVHNAKTKP
REEQYNSTYRVVSVLTVLHQDWLNGKEYKCKVSNKALPLPIEKTISKAK
SEQ ID NO. 9 (= Fc ALIE region (IgG1-C_{H}2.A330L.I332E))
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPLPEEKTISKAK
SEQ ID NO. 10 (= Fc GASD region (IgG1-C_{H}2.G236A.S239D))
APELLAGPDVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
SEQ ID NO. 11 (= Fc GASDALIE region (IgG1-C_{H}2.G236A.S239D.A330L.I332E))
APELLAGPDVFLFPPKPKDTLMISRTPEVTCVVVDVSHEDPEVKENWYVDGVEVHNAKTKP
REEQYNSTYRVVSVLTVLHQDWLNGKEYKCKVSNKALPLPEEKTISKAK
SEQ ID NO. 12 (= Fc GASDIE region (IgG1-C_{H}2.G236A.S239D.I332E))
APELLAGPDVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPEEKTISKAK
SEQ ID NO. 13 (= Fc SDALIE region (IgG1-C_{H}2.S239D.A330L.I332E))
APELLGGPDVFLFPPKPKDTLMISRTPEVTCVVVDVSHEDPEVKFNWYVDGVEVHNAKTKP
REEQYNSTYRVVSVLTVLHQDWLNGKEYKCKVSNKALPLPEEKTISKAK
SEQ ID NO. 14 (= Fc LALA region (IgG1-C_{H}2.L234A.L235A))
APEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
SEQ ID NO. 15 (= Fc WT region (IgG1) allotype nG1m1)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO. 16 (= Fc WT region (IgG1) allotype G1m17,1)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO. 17 (= Fc WT region (IgG1) allotype G1m17,1 encompassed in SEQ ID NO. 16)
CPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK

1. A composition comprising a CD16+ cell in combination with a recombinant polypeptide capable of binding to the FcγRIII (CD16) surface protein, wherein the recombinant polypeptide is non-covalently bound to the FcγRIII (CD16) surface protein expressed by the CD16+ cell, and wherein said recombinant polypeptide comprises:
(i) a modified C_{H}2 domain of a wild-type human IgG1, bound, optionally through a linker, to
(ii) a ligand binding domain,
wherein the ligand binding domain comprises a sequence capable of binding to a target ligand;
wherein the modified C_{H}2 domain is characterized by comprising mutations S239D and I332E with respect to the C_{H}2 domain of a wild-type human IgG1, and wherein said C_{H}2 domain of a wild-type human IgG1 is represented by SEQ ID NO 1, or by a sequence with a percentage of identity of at least 85% with the amino acid sequence SEQ ID NO. 1, and comprises sequence positions 231-340, according to the EU numbering,

2. The composition of claim 1, wherein the modified C_{H}2 domain is modified with respect to the C_{H}2 domain of the wild-type human IgG1 represented by SEQ ID NO 1.

3. The composition of claim 1, wherein the FcγRIII (CD16) surface protein is FcγRIIIa/CD16a surface protein.

4. The composition of claim 2, wherein the modified C_{H}2 domain comprises at least one additional mutation selected from the list consisting of H268F, S324T, G236A and A330L with respect to the C_{H}2 domain of a wild-type human IgG1.

5. The composition of claim 2, wherein
(a) the modified C_{H}2 domain comprises at least one additional mutation selected from the list consisting of H268F, S324T and A330L with respect to the C_{H}2 domain of a wild-type human IgG1; or
(b) the modified C_{H}2 domain comprises at least one additional mutation selected from the list consisting of H268F and S324T with respect to the C_{H}2 domain of a wild-type human IgG1.

6. (canceled)

7. The composition of claim 5, wherein
(I) when the modified C_{H}2 domain comprises at least one additional mutation selected from the list consisting of H268F and S324T with respect to the C_{H}2 domain of a wild-type human IgG1, then (A) the modified C_{H}2 domain further comprises amino acid substitutions S239D, I332E and S324T, according to EU numbering, with respect to the C_{H}2 domain of a wild-type human IgG1, (B) the modified C_{H}2 domain further comprises amino acid substitutions S239D, I332E and H268F, according to EU numbering, with respect to the C_{H}2 domain of a wild-type human IgG1, or (C) the modified C_{H}2 domain further comprises amino acid substitutions S239D, I332E, H268F and S324T, according to EU numbering, with respect to the C_{H}2 domain of a wild-type human IgG1; or
(II) the modified C_{H}2 domain comprises at least one additional mutation selected from the list consisting of H268F, S324T and A330L with respect to the C_{H}2 domain of a wild-type human IgG1, then the modified C_{H}2 domain further comprises amino acid substitutions S239D, I332E and A330L, according to EU numbering, with respect to the C_{H}2 domain of a wild-type human IgG1.

8.-10. (canceled)

11. The composition of claim 2, wherein the modification of the C_{H}2 domain with respect to the C_{H}2 domain of a wild-type human IgG1 consists of mutations S239D and I332E.

12. The composition of claim 1, wherein the CD16+ cell is allogeneic with respect to an individual in need thereof.

13. The composition of claim 1, where the recombinant polypeptide is an antibody and the modification in the C_{H}2 domain is symmetrical, or asymmetrical, with respect to the pair of C_{H}2 domains (or the pair of heavy chains) constituting the antibody.

14. The composition of claim 1, wherein the recombinant polypeptide comprises a human IgG1 Fc (fragment crystallizable) region comprising the modified C_{H}2 domain.

15. The composition according to claim 1, wherein the recombinant polypeptide is an antibody or a fragment thereof comprising the modified C_{H}2 domain as defined in any of claims 1 to 6 or the Fc region as defined in claim 14 and a ligand binding domain selected from the ligand binding domain of any of the following antibodies: Abagovomab, Abatacept, Abciximab, Abituzumab, Abrilumab, Actoxumab, Adalimumab, Adecatumab, Aducanumab, Aflibercept, Afutuzymab, Alacizumab, Alefacept, Alemtuzumab, Alirocumab, Altumomab, Amatixumab, Anatumomab, Anetumab, Anifromumab, Anrukinzumab, Apolizumab, Arcitumomab, Ascrinvacumab, Aselizumab, Atezolizumab, Atinumab, Altizumab, Atorolimumab, Bapineuzumab, Basiliximab, Bavituximab, Bectumomab, Begelomab, Belatacept, Belimumab, Benralizumab, Bertilimumab, Besilesomab, Bevacizumab, Bezlotoxumab, Biciromab, Bimagrumab, Bimekizumab, Bivatuzumab, Blinatumomab, Blosozumab, Bococizumab, Brentuximab, Briakimumab, Brodalumab, Brolucizumab, Bronticizumab, Canakinumab, Cantuzumab, Caplacizumab, Capromab, Carlumab, Catumaxomab, Cedelizumab, Certolizumab, Cetixumab, Citatuzumab, Cixutumumab, Clazakizumab, Clenoliximab, Clivatuzumab, Codrituzumab, Coltuximab, Conatumumab, Concizumab, Crenezumab, Dacetuzumab, Daclizumab, Dalotuzumab, Dapirolizumab, Daratumumab, Dectrekumab, Demcizumab, Denintuzumab, Denosumab, Derlotixumab, Detumomab, Dinutuximab, Diridavumab, Dorlinomab, Drozitumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab, Enlimomab, Enoblituzumab, Enokizumab, Enoticumab, Ensituximab, Epitumomab, Epratuzomab, Erlizumab, Ertumaxomab, Etanercept, Etaracizumab, Etrolizumab, Evinacumab, Evolocumab, Exbivirumab, Fanolesomab, Faralimomab, Farletuzomab, Fasimumab, Felvizumab, Fezkimumab, Ficlatuzumab, Figitumumab, Firivumab, Flanvotumab, Fletikumab, Fontolizumab, Foralumab, Foravirumab, Fresolimumab, Fulramumab, Futuximab, Galiximab, Ganitumab, Gantenerumab, Gavilimomab, Gemtuzumab, Gevokizumab, Girentuximab, Glembatumumab, Golimumab, Gomiliximab, Guselkumab, Ibalizumab, Ibritumomab, Icrucumab, Idarucizumab, Igovomab, Imalumab, Imciromab, Imgatuzumab, Inclacumab, Indatuximab, Indusatumab, Infliximab, Intetumumab, Inolimomab, Inotuzumab, Ipilimumab, Iratumumab, Isatuximab, Itolizumab, Ixekizumab, Keliximab, Labetuzumab, Lambrolizumab, Lampalizumab, Lebrikizumab, Lemalesomab, Lenzilumab, Lerdelimumab, Lexatumumab, Libivirumab, Lifastuzumab, Ligelizumab, Lilotomab, Lintuzumab, Lirilumab, Lodelcizumab, Lokivetmab, Lorvotuzumab, Lucatumumab, Lulizumab, Lumiliximab, Lumretuzumab, Mapatumumab, Margetuximab, Maslimomab, Mavrilimumab, Matuzumab, Mepolizumab, Metelimumab, Milatuzumab, Minetumomab, Mirvetuximab, Mitumomab, Mogamulizumab, Morolimumab, Motavizumab, Moxetumomab, Muromonab-CD3, Nacolomab, Namilumab, Naptumomab, Narnatumab, Natalizumab, Nebacumab, Necitumumab, Nemolizumab, Nerelimomab, Nesvacumab, Nimotuzumab, Nivolumab, Nofetumomab, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab, Oregovomab, Orticumab, Otelixizumab, Oltertuzumab, Oxelumab, Ozanezumab, Ozoralizumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab, Pintumomab, Polatuzumab, Ponezumab, Priliximab, Pritumumab, Quilizumab, Racotumomab, Radretumab, Rafivirumab, Ralpancizumab, Ramucirumab, Ranibizumab, Raxibacumab, Refanezumab, Regavirumab, Reslizumab, Rilonacept, Rilotumumab, Rinucumab, Rituximab, Robatumumab, Roledumab, Romosozumab, Rontalizumab, Rovelizumab, Ruplizumab, Sacituzumab, Samalizumab, Sarilumab, Satumomab, Secukimumab, Seribantumab, Setoxaximab, Sevirumab, Sibrotuzumab, Sifalimumab, Siltuximab, Siplizumab, Sirukumab, Sofituzumab, Solanezumab, Solitomab, Sonepcizumab, Sontuzumab, Stamulumab, Sulesomab, Suvizumab, Tabalumab, Tacatuzumab, Tadocizumab, Talizumab, Tanezumab, Taplitumomab, Tarextumab, Tefibazumab, Telimomab aritox, Tenatumomab, Teneliximab, Teplizumab, Tesidolumab, TGN 1412, Ticlimumab, Tildrakizumab, Tigatuzumab, TNX-650, Tocilizumab, Toralizumab, Tosatoxumab, Tositumomab, Tovetumab, Tralokimumab, Trastuzumab, TRBS07, Tregalizumab, Tremelimumab, Trevogrumab, Tucotuzumab, Tuvirumab, Ublituximab, Ulocuplumab, Urelumab, Urtoxazumab, Ustekimumab, Vandortuzumab, Vantictumab, Vanucizumab, Vapaliximab, Varlimumab, Vatelizumab, Vedolizumab, Veltuzumab, Vepalimomab, Vesencumab, Visilizumab, Volocixumab, Vorsetuzumab, Votumumab, Zalutumimab, Zanolimumab, Zatuximab, Ziralimumab, Ziv-Aflibercept, and Zolimomab.

16. A method for treating or preventing a cancer, an autoimmune disease, or an infectious disease in an individual in need thereof comprising administering the composition of claim 1 to said individual.

17. A pharmaceutical composition comprising the composition according to claim 1, further comprising an excipient or a pharmacologically acceptable vehicle.

18. (canceled)

19. A composition comprising a NK (Natural Killer) cell in combination with a recombinant polypeptide capable of binding to the FcγRIII (CD16) surface protein, wherein the recombinant polypeptide is non-covalently bound to the FcγRIII (CD16) surface protein expressed by the NK (Natural Killer) cell, and wherein said recombinant polypeptide comprises:
(iii) a modified C_{H}2 domain of a wild-type human IgG1, bound, optionally through a linker, to
(iv) a ligand binding domain,
wherein the ligand binding domain comprises a sequence capable of binding to a target ligand;
wherein the modified C_{H}2 domain is characterized by comprising mutations S239D and I332E with respect to the C_{H}2 domain of a wild-type human IgG1, and wherein said C_{H}2 domain of a wild-type human IgG1 is represented by SEQ ID NO. 1, or by a sequence with a percentage of identity of at least 85% with the amino acid sequence SEQ ID NO. 1, and comprises sequence positions 231-340, according to the EU numbering,

20.-32. (canceled)

33. A method for treating or preventing a cancer, an autoimmune disease, or an infectious disease in an individual in need thereof comprising administering the composition of claim 19 to said individual.

34. A pharmaceutical composition comprising the composition according to claim 19, further comprising an excipient or a pharmacologically acceptable vehicle.

35. (canceled)

36. Pharmaceutical composition comprising a NK (Natural Killer) cell, in combination with a recombinant polypeptide; wherein said recombinant polypeptide comprises (i) a modified Fc (fragment crystallizable) region, and (ii) a ligand binding domain, said Fc region being capable of binding to said NK cell and comprising at least one modified C_{H}2 domain of a wild-type human IgG1, wherein the modified C_{H}2 domain is characterized by comprising mutations S239D and I332E with respect to the C_{H}2 domain of a wild-type human IgG1, and wherein said C_{H}2 domain of a wild-type human IgG1 is represented by SEQ ID NO 1, or by a sequence with a percentage of identity of at least 85% with the amino acid sequence SEQ ID NO. 1, and comprises sequence positions 231-340, according to the EU numbering.

37.-47. (canceled)

48. A NK cell allogeneic with respect to an individual in need thereof attached to a recombinant polypeptide, wherein said recombinant polypeptide comprises (i) a modified Fc (fragment crystallizable) region, and (ii) a binding domain, said Fc region being capable of binding to said NK cell and comprising at least one modified C_{H}2 domain of a wild-type human IgG1 as defined in claim 36.

49. A kit comprising:
a first part comprising a CD16+ cell, preferably a CD16+ cell allogeneic with respect to an individual in need thereof; and
a second part comprising a recombinant polypeptide as defined in claim 1.

50. A kit comprising:
a first part comprising a NK cell, preferably a NK cell allogeneic with respect to an individual in need thereof; and
a second part comprising a recombinant polypeptide as defined in claim 19.

51. A Kit comprising:
a first part including an NK cell, preferably an NK cell allogeneic with respect to an individual in need thereof; and
a second part including a recombinant polypeptide comprising (i) a modified Fc (fragment crystallizable) region, and (ii) a ligand binding domain; said Fc region being capable of binding to said NK cell and comprising at least one modified C_{H}2 domain of a wild-type human IgG1 as defined in claim 36.

52. Pharmaceutical composition comprising a CD16+ cell, in combination with a recombinant polypeptide; wherein said recombinant polypeptide comprises (i) a modified Fc (fragment crystallizable) region on the amino acid sequence of the Fc region, and (ii) a ligand binding domain, said Fc region being capable of binding to said CD16+ cell, and further comprising an excipient or a pharmacologically acceptable vehicle.

53. The pharmaceutical composition of claim 52, wherein the recombinant polypeptide is non-covalently bound to the CD16+ cell.

54. Pharmaceutical composition comprising a NK (Natural Killer) cell or a NK cell precursor, in combination with a recombinant polypeptide; wherein said recombinant polypeptide comprises (i) a modified Fc (fragment crystallizable) region on the amino acid sequence of the Fc region, and (ii) a ligand binding domain, said Fc region being capable of binding to said NK cell or precursor thereof, and further comprising an excipient or a pharmacologically acceptable vehicle.

55. The pharmaceutical composition of claim 54, wherein the recombinant polypeptide is non-covalently bound to the NK cell.


SEQUENCE LISTING (17 sequences)

SEQ ID NO: 1 - protein, 110 aa, Artificial Sequence
  region Fc WT
  region Fc WT
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 2 - protein, 110 aa, Artificial Sequence
  region Fc SDH
  region Fc SDH
APELLGGPDVFLFPPKPKDTLMISRTPEVTCVVVDVSFEDPEVKFNWYVDGVEVHNAKTKPREEQYNSTYRVVSVLTVLHQDWLNGKEYKCKVTNKALPAPEEKTISKAK

SEQ ID NO: 3 - protein, 110 aa, Artificial Sequence
  region Fc SD
  region Fc SD
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 4 - protein, 110 aa, Artificial Sequence
  region Fc IE
  region Fc IE
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAK

SEQ ID NO: 5 - protein, 110 aa, Artificial Sequence
  region Fc SDIE
  region Fc SDIE
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAK

SEQ ID NO: 6 - protein, 110 aa, Artificial Sequence
  region Fc SDSTIE
  region Fc SDSTIE
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVTNKALPAPEEKTISKAK

SEQ ID NO: 7 - protein, 110 aa, Artificial Sequence
  region Fc SDHFIE
  region Fc SDHFIE
APELLGGPDVFLFPPKPKDTLMISRTPEVTCVVVDVSFEDPEVKFNWYVDGVEVHNAKTKPREEQYNSTYRVVSVLTVLHQDWLNGKEYKCKVSNKALPAPEEKTISKAK

SEQ ID NO: 8 - protein, 110 aa, Artificial Sequence
  region Fc AL
  region Fc AL
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAK

SEQ ID NO: 9 - protein, 110 aa, Artificial Sequence
  region Fc ALIE
  region Fc ALIE
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 10 - protein, 110 aa, Artificial Sequence
  region Fc GASD
  region Fc GASD
APELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 11 - protein, 110 aa, Artificial Sequence
  region Fc GASDALIE
  region Fc GASDALIE
APELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 12 - protein, 110 aa, Artificial Sequence
  region Fc GASDIE
  region Fc GASDIE
APELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAK

SEQ ID NO: 13 - protein, 110 aa, Artificial Sequence
  region Fc SDALIE
  region Fc SDALIE
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 14 - protein, 110 aa, Artificial Sequence
  region Fc LALA
  region Fc LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAK

SEQ ID NO: 15 - protein, 232 aa, Artificial Sequence
  “Fc WT region (IgG1) allotype nG1m1”
  Fc WT region (IgG1) allotype nG1m1
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 232 aa, Artificial Sequence
  “Fc WT region (IgG1) allotype G1m17,1”
  Fc WT region (IgG1) allotype G1m17,1
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 222 aa, Artificial Sequence
  “Fc WT region (IgG1) allotype G1m17,1”
  Fc WT region (IgG1) allotype G1m17,1
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK